(19) United States
(12) Patent Application Publication
(10) Pub. No.: US 2025/0148601 A1
(43) Pub. Date: May 8, 2025

(54) SIMULATING STRUCTURES IN IMAGES

(71) Applicant: Hyperfine Operations, Inc., Guilford, CT (US)

(72) Inventors: Michal Sofka, Princeton, NJ (US); Jo Schlemper, Long Island City, NY (US)

(73) Assignee: Hyperfine Operations, Inc., Guilford, CT (US)

(21) Appl. No.: 19/017,224

(22) Filed: Jan. 10, 2025

Related U.S. Application Data
(63) Continuation of application No. PCT/US2023/027537, filed on Jul. 12, 2023.
(60) Provisional application No. 63/388,995, filed on Jul. 13, 2022.

(51) Int. Cl. G06T 7/00; G01R 33/56; G06T 7/11; G06T 7/187; G06T 7/73; G06T 11/00; G06T 19/20

(52) U.S. Cl. CPC G06T 7/0012; G01R 33/5608; G06T 7/11; G06T 7/187; G06T 7/74; G06T 11/008; G06T 19/20; G06T 2207/10088; G06T 2207/20081; G06T 2207/20104; G06T 2207/20152; G06T 2207/30016; G06T 2207/30096; G06T 2210/41; G06T 2219/2021

(57) ABSTRACT

Systems and methods for simulating structures and images are disclosed. The techniques described herein can include obtaining a first image of a subject. The techniques can include determining a location for simulating a structure within the first image. The techniques can include simulating, according to the location, a shape for the structure. The techniques can include generating a mask according to the location and the shape for the structure. The techniques can include applying the mask to the first image to generate a second image simulating the structure.

### CROSS-REFERENCE TO RELATED APPLICATIONS

This application is a bypass continuation of international application PCT/US2023/027537 filed Jul. 12, 2023, which claims priority to U.S. Provisional Application No. 63/388,995 filed Jul. 13, 2022, titled “Abnormality Simulation Improves Model Performance,” each of which is incorporated herein by reference in its entirety.

### FIELD OF THE DISCLOSURE

The present invention relates generally to the field of medical imaging, including multi-shot magnetic resonance (MR) imaging.

### BACKGROUND

Magnet resonance imaging (MRI) systems may be utilized to generate images of the inside of the human body. MRI systems may be used to detect magnetic resonance (MR) signals in response to applied electromagnetic fields. MRI techniques may include fast spin-echo (FSE) imaging, in which a series of radio frequency (RF) pulses are used to excite the protons in tissues. FSE imaging techniques are used to construct images from multiple echoes, which are generated by refocusing the magnetization of the excited protons, to increase the speed of image acquisition.

### SUMMARY

The systems and methods of this technical solution provide techniques for simulating structures and/or images. By utilizing the techniques described herein, one or more structures, such as normal structures (e.g., healthy tissues) and/or abnormal structures (e.g., lesions), can be simulated for at least one body part of a subject (e.g., a patient or an individual). The body part may include, but is not limited to, a brain, heart, lung, liver, kidneys, or other parts of the body. The structures can be applied to images of various body parts associated with one or more subjects. In some aspects, the techniques can simulate one or more images by applying the simulated structures to at least one historical image or aggregating the simulated structures to simulate/generate at least one new image. By utilizing these techniques, the simulated structures and/or images can be used for training a machine learning (ML) or artificial intelligence (AI) model (e.g., sometimes referred to as an ML/AI model) to improve model performance.

At least one aspect of the present disclosure is directed to a method for simulating structures in images. The method can include obtaining a first image of a subject. The method can include determining a location for simulating a structure within the first image. The method can include simulating, according to the location, a shape for the structure. The method can include generating a mask according to the location and the shape for the structure. The method can include applying the mask to the first image to generate a second image simulating the structure.

In some implementations, to determine the location within the image, the method can include identifying at least one anatomical region associated with a body part of the subject. The method can include extracting, using the identified at least one anatomical region, a plurality of territories associated with the first image. The method can include selecting at least one first territory associated with the first image as the location for the mask.

In some implementations, subsequent to selecting the at least one first territory, the method can include selecting at least one second territory associated with the first image as a second location for another mask for generating a third image. In some implementations, to determine the location and simulating the shape, the method can include receiving an indication of at least one territory associated with the first image as the location. The method can include simulating the shape of the structure based on the at least one territory associated with the first image.

In some implementations, to generate the mask, the method can include providing a seed to the first image at the location. The method can include growing at least one region around the seed using at least one region-growing algorithm. The method can include generating the mask based on the grown seed. In some implementations, the at least one region-growing algorithm can comprise at least one of: region growing, region merging, split and merge, watershed transform, connected component labeling, or graph-cut segmentation.

In some implementations, the seed can be provided at at least one voxel having a first intensity. The region around the seed may be grown to at least one neighboring voxel having a second intensity, wherein the second intensity is substantially similar to the first intensity. In some implementations, to simulate the shape, the method can include generating an elliptical shape according to one or more parameters, the one or more parameters comprising a long axis or a short axis of the first image defining a dimension of the structure. The method can include applying an elastic distortion to the elliptical shape to simulate the shape of the structure.

In some implementations, to generate the mask, the method can include obtaining a plurality of historical masks associated with a plurality of images from one or more subjects. The method can include training a model using at least one machine learning technique based on the plurality of historical masks. The method can include generating, using the trained model, the mask for the location according to the plurality of historical masks. The method can include refining the mask based on a comparison between the generated mask and the plurality of historical masks associated with the location for simulating the structure.

In some implementations, to generate the mask, the method can include determining an appearance of the mask associated with at least an intensity of one or more voxels of the first image. In some implementations, to determine the appearance, the method can include selecting an aggregated pixel intensity of the mask. The method can include applying at least one pattern for the mask. The method can include simulating at least one noise for the mask. In some implementations, the at least one pattern can comprise at least one of: edema pattern, hemorrhagic pattern, necrotic pattern, cystic pattern, inflammatory pattern, tumoral pattern, or ischemic pattern.

In some implementations, to determine the appearance and apply the mask, the method can include providing the generated mask and at least a portion of the first image for applying the mask as inputs to a model trained using a machine learning technique. The method can include generating, using the model, a third image comprising at least the portion of the first image and the generated mask. The method can include updating the third image based on a comparison of the third image to at least one historical image, the at least one historical image having a second mask at the location with a second shape similar to the shape of the mask.

In some implementations, to apply the mask, the method can include simulating a mass effect around the mask. The method can include applying the mask to the first image with the simulated mass effect to generate the second image simulating the structure. In some implementations, the first image can be a first magnetic resonance (MR) image and the second image can be a second MR image.

In some implementations, to generate the mask, the method can include simulating a shape deformation associated with the shape. The method can include generating the mask according to the location and the shape deformation for the structure. In some implementations, simulating the shape deformation may correspond to simulating hydrocephalus for the body part of the subject.

In some implementations, to apply the mask, the method can include identifying a plurality of anatomical regions associated with a body part of the subject. The method can include simulating contrast for the first image based on at least one of the plurality of anatomical regions and one or more sequence parameters. The method can include applying the mask and the simulated contrast to the first image to generate the second image simulating the structure.

In some implementations, the one or more sequence parameters can comprise at least one of relaxation time, echo time, inversion time, or flip angle. In some implementations, each of the plurality of anatomical regions is represented as a 2-dimensional (2D) image or a 3-dimensional (3D) image. In some implementations, the contrast may indicate at least one of gray matter (GM), white matter (WM), or cerebrospinal fluid (CSF) associated with the body part of the subject.

In some implementations, to simulate the contrast, the method can include using at least one signal equation, comprising at least one of: spin-echo, spoiled gradient echo, rewound gradient, time-reversed gradient echo, or random. In some implementations, to generate the mask, the method can include determining an appearance of the mask based on the simulated contrast.

In some implementations, the method can include obtaining a plurality of images of a plurality of subjects, comprising at least a fourth image and a fifth image associated with a body part of at least one subject. The method can include conforming the fifth image to the fourth image. The method can include simulating a sixth image according to the conformed fifth image.

In some implementations, to conform the fifth image to the fourth image the method can include at least one of: conforming a first contrast of the fifth image to a second contrast of the fourth image; and conforming a first size of the fifth image to a second size of the fourth image. In some implementations, the fourth image can be a neonatal image of the body part of the at least one subject, the fifth image can be a developed image of the body part of the at least one subject, and the sixth image can be another neonatal image simulated from the developed image of the body part of the at least one subject.

In some implementations, to conform the first contrast to the second contrast and conform the first size to the second size the method can comprise at least one of: injecting one or more shapes to the fifth image according to the fourth image; overriding one or more values associated with pixels or voxels of the fifth image according to the fourth image; or applying one or more distortions to the fifth image according to the fourth image.

At least one other aspect of the present disclosure is directed to a system for simulating structures in images. The system can include one or more processors configured to obtain a first image of a subject. The one or more processors can determine a location for simulating a structure within the first image. The one or more processors can simulate, according to the location, a shape for the structure. The one or more processors can generate a mask according to the location and the shape for the structure. The one or more processors can apply the mask to the first image to generate a second image simulating the structure.

Yet another aspect of the present disclosure is directed to non-transitory computer-readable storage medium storing processor-executable instructions that, when executed by at least one processor, cause the at least one processor to obtain a first image of a subject; determine a location for simulating a structure within the first image; simulate, according to the location, a shape for the structure; generate a mask according to the location and the shape for the structure; and apply the mask to the first image to generate a second image simulating the structure.

These and other aspects and implementations are discussed in detail below. The foregoing information and the following detailed description include illustrative examples of various aspects and implementations, and provide an overview or framework for understanding the nature and character of the claimed aspects and implementations. The drawings provide illustration and a further understanding of the various aspects and implementations, and are incorporated in and constitute a part of this specification. Aspects may be combined and it will be readily appreciated that features described in the context of one aspect of the present disclosure may be combined with other aspects. Aspects may be implemented in any convenient form. In a non-limiting example, by appropriate computer programs, which may be carried on appropriate carrier media (computer readable media), which may be tangible carrier media (e.g. disks) or intangible carrier media (e.g. communications signals). Aspects may also be implemented using suitable apparatus, which may take the form of programmable computers running computer programs arranged to implement the aspect. As used in the specification and in the claims, the singular form of “a,” “an,” and “the” include plural referents unless the context clearly dictates otherwise.

### BRIEF DESCRIPTION OF THE DRAWINGS

The accompanying drawings are not intended to be drawn to scale. Like reference numbers and designations in the various drawings indicate like elements. For purposes of clarity, not every component may be labeled in every drawing. The patent or application file contains at least one drawing executed in color. Copies of this patent or patent application publication with color drawing(s) will be provided by the Office upon request and payment of the necessary fee. In the drawings:

FIG. 1 illustrates example components of a magnetic resonance imaging system, which may be utilized to implement the techniques to simulate structures or images, in accordance with one or more implementations;

FIG. 2 depicts an example pipeline for simulating structures for at least one image, in accordance with one or more implementations;

FIG. 3 depicts an example of brain territory classification, in accordance with one or more implementations;

FIG. 4 depicts an example of at least one territory selected as a targeted location of a simulated lesion, in accordance with one or more implementations;

FIG. 5 depicts examples of generated lesion masks of different shapes and sizes, in accordance with one or more implementations;

FIG. 6 depicts example graphs for volume distributions of real lesions and simulated lesions with lesion transfer, in accordance with one or more implementations;

FIG. 7 depicts an example of 2D slices including a generated lesion for interpolation, in accordance with one or more implementations;

FIG. 8 depicts an example bar graph for the intensity distribution of lesions on diffusion-weighted imaging (DWI), in accordance with one or more implementations;

FIG. 9 depicts an example of a simulated edema effect around a lesion, in accordance with one or more implementations;

FIG. 10 depicts example images of acquisition noise in the simulated lesion, in accordance with one or more implementations;

FIG. 11 depicts an example of different noise levels for the image, in accordance with one or more implementations;

FIG. 12 depicts an example of a simulated mass effect around a mask, in accordance with one or more implementations;

FIG. 13 depicts an example of a hydrocephalus simulation, in accordance with one or more implementations;

FIG. 14 depicts an example of MR images with different contrast, in accordance with one or more implementations;

FIG. 15 depicts an example of tissue region probability map for gray matter (GM), white matter (WM), and cerebrospinal fluid (CSF), in accordance with one or more implementations;

FIG. 16 depicts an example of the same brain using a spin echo signal equation and generating different contrast according to different repetition time (TR), echo time (TE), and/or inversion time (TI), in accordance with one or more implementations;

FIG. 17 depicts an example of contrast generated using random signal equation, in accordance with one or more implementations;

FIG. 18 depicts an example of pediatrics simulations, in accordance with one or more implementations;

FIG. 19 depicts a flowchart of an example method for simulating structures or images, in accordance with one or more implementations; and

FIG. 20 is a block diagram of an example computing system suitable for use in the various arrangements described herein, in accordance with one or more example implementations.

### DETAILED DESCRIPTION

Below are detailed descriptions of various concepts related to and implementations of techniques, approaches, methods, apparatuses, and systems for suppressing artifacts in multi-shot FSE imaging. The various concepts introduced above and discussed in detail below may be implemented in any of numerous ways, as the described concepts are not limited to any particular manner of implementation. Examples of specific implementations and applications are provided primarily for illustrative purposes.

Imaging techniques, such as magnetic resonance imaging (MRI) techniques, computed tomography (CT) imaging techniques, ultrasound imaging techniques, etc., may be utilized in clinical practice for scanning patients/subjects. When utilized, these imaging techniques can produce or generate images depicting aspects of the scanned portions (e.g., body parts) of the subjects. These aspects may include soft tissues and/or hard tissues. For example, taking MR imaging as an example, a magnetic field and radio waves series can be used to create detailed images of the body's internal structures. The operation of MR imaging may include aligning the hydrogen atoms in the body with the magnetic field and applying radio waves to excite these atoms. As the atoms return to their original alignment, radio signals are emitted, which can be detected by the MRI machine/system for processing (e.g., to generate cross-sectional images). Some uses of MR imaging can include imaging the brain, spine, or joints (among other body parts), or identifying lesions or abnormalities in soft tissues. These images provide information that is useful for diagnosing and treating a variety of health conditions.

These images are also useful for training or improving the performance of ML (or AI) models (e.g., sometimes referred to generally as models) to identify various aspects of the body parts, including but not limited to lesion detection or analysis. For instance, in certain systems, the model can be trained to detect lesions (or other abnormalities) based on, or by utilizing, historical images including examples of normal cases and abnormal cases (e.g., body parts with lesions). These historical images can be provided as training datasets for the model. During training, the ML model can learn the patterns or features distinguishing the lesions from healthy tissue. The model may use any suitable type of ML technique for training and execution, such as convolutional neural networks (CNNs), support vector machines (SVMs), clustering algorithms, etc., to extract relevant features, label lesions within the images, or other operations for diagnosing the subject.

To improve the performance of the models, a large number of training datasets may be desired. However, obtaining real-world datasets (e.g., actual scanned images of the subject) for training the model may consume excessive resources and time, with certain characteristics (e.g., locations, shapes, or appearances) of a lesion being seldom or rarely encountered for analytic or training purposes. Further, these new images from the imaging device/machine may be labeled manually (e.g., by technicians or healthcare providers) or automatically by an existing model, which may introduce erroneous training datasets, including labeling a portion of the healthy tissue as a lesion or a portion of a lesion as a healthy tissue, for example.

Hence, the systems and methods of the technical solution discussed herein can provide techniques for simulating structures or images for improving model performance. By utilizing the techniques discussed herein, the systems and methods can simulate structures of healthy tissues and abnormalities for images of various body parts. For example, the techniques described herein can simulate healthy tissues and lesions for images of at least one part of the body. The techniques described herein can simulate one or multiple lesions within an image. Although aspects of this disclosure are described in connection with MRI (e.g., MR images), it should be understood that the techniques described herein can be used to simulate the structures for other types of imaging, such as but not limited to CT images, ultrasound images, or positron emission tomography (PET) scan images. For purposes of providing examples herein, the body part of the subject may refer to the brain, although images or structures of other body parts can be simulated utilizing similar techniques discussed herein, including but not limited to the brains, lungs, livers, kidneys, etc.

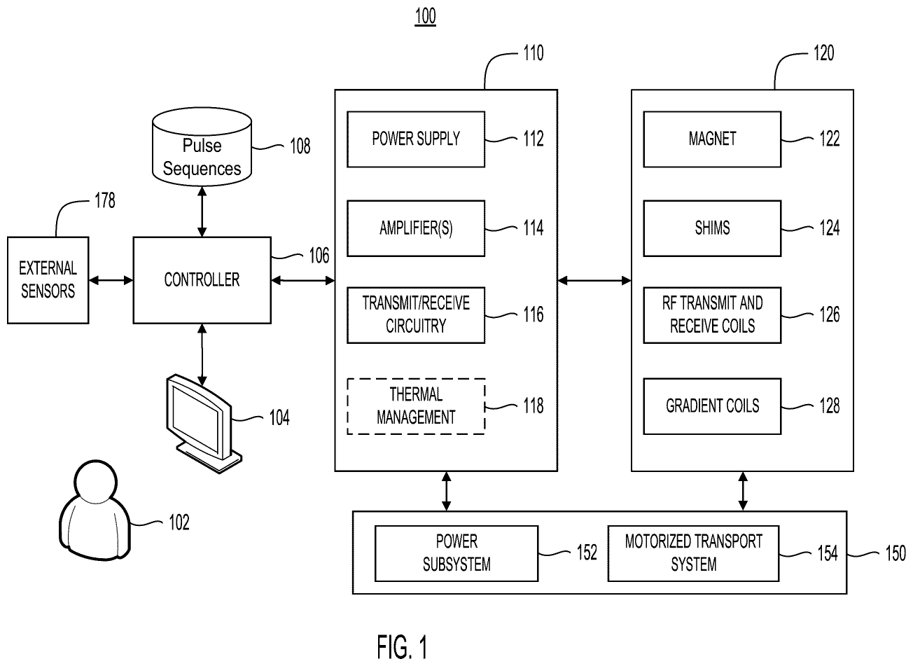

FIG. 1 illustrates an example MRI system which may be utilized in connection with the structure simulation techniques described herein. In FIG. 1, MRI system 100 may include a computing device 104, a controller 106, a pulse sequences repository 108, a power management system 110, and magnetics components 120. The MRI system 100 is illustrative, and an MRI system may have one or more other components of any suitable type in addition to or instead of the components illustrated in FIG. 1. In some implementations, the one or more components of the MRI system 100 may be operated by a user 102 or other authorized personnel, including direct or remote operation. Additionally, the implementation of components for a particular MRI system may differ from those described herein. Examples of low-field MRI systems may include portable MRI systems, which may have a field strength that may be, in a non-limiting example, less than or equal to 0.5 T, that may be less than or equal to 0.2 T, that may be within a range from 1 mT to 100 mT, that may be within a range from 50 mT to 0.1 T, that may be within a range of 40 mT to 80 mT, that may be about 64 mT, etc.

The magnetics components 120 may include B₀ magnets 122, shims 124, RF transmit and receive coils 126, and gradient coils 128. The B₀ magnets 122 may be used to generate a main magnetic field B₀. B₀ magnets 122 may be any suitable type or combination of magnetics components that may generate a desired main magnetic B₀ field. In some embodiments, B₀ magnets 122 may be one or more permanent magnets, one or more electromagnets, one or more superconducting magnets, or a hybrid magnet comprising one or more permanent magnets and one or more electromagnets or one or more superconducting magnets. In some embodiments, B₀ magnets 122 may be configured to generate a B₀ magnetic field having a field strength that may be less than or equal to 0.2 T or within a range from 50 mT to 0.1 T.

In some implementations, the B₀ magnets 122 may include a first and second B₀ magnet, which may each include permanent magnet blocks arranged in concentric rings about a common center. The first and second B₀ magnet may be arranged in a bi-planar configuration such that the imaging region is located between the first and second B₀ magnets. In some embodiments, the first and second B₀ magnets may each be coupled to and supported by a ferromagnetic yoke configured to capture and direct magnetic flux from the first and second B₀ magnets.

The gradient coils 128 may be arranged to provide gradient fields and, in a non-limiting example, may be arranged to generate gradients in the B₀ field in three substantially orthogonal directions (X, Y, and Z). Gradient coils 128 may be configured to encode emitted MR signals by systematically varying the B₀ field (the B₀ field generated by the B₀ magnets 122 or shims 124) to encode the spatial location of received MR signals as a function of frequency or phase. In a non-limiting example, the gradient coils 128 may be configured to vary frequency or phase as a linear function of spatial location along a particular direction, although more complex spatial encoding profiles may also be provided by using nonlinear gradient coils. In some embodiments, the gradient coils 128 may be implemented using laminate panels (e.g., printed circuit boards), in a non-limiting example.

During FSE scans, the gradient coils 128 may be controlled to produce phase encoding gradients, which may be used to sample an MR signal at different positions along different directions (e.g., the X, Y, and Z orthogonal directions). The phase-encoding gradient is a magnetic field gradient that varies linearly along the phase-encoding direction, which causes a phase shift in the MR signal according to its position in that direction. The gradient coils 128 can be controlled (e.g., by the controller 106) to vary the amplitude of the phase-encoding gradient for each acquisition, causing different locations along the phase-encoding direction to be sampled. The MR signal can be spatially encoded in the phase-encoding direction. The number of phase encoding steps can influence the resolution of the MR image along the phase-encoding direction. For example, increasing the number of phase encoding steps may improve image resolution but also increases scan time, while decreasing the number of phase encoding steps may reduce image resolution and decreases scan time.

MRI scans are performed by exciting and detecting emitted MR signals using transmit and receive coils, respectively (referred to herein as radio frequency (RF) coils). The transmit and receive coils may include separate coils for transmitting and receiving, multiple coils for transmitting or receiving, or the same coils for transmitting and receiving. Thus, a transmit/receive component may include one or more coils for transmitting, one or more coils for receiving, or one or more coils for transmitting and receiving. The transmit/receive coils may be referred to as Tx/Rx or Tx/Rx coils to generically refer to the various configurations for transmit and receive magnetics components of an MRI system. These terms are used interchangeably herein. In FIG. 1, RF transmit and receive coils 126 may include one or more transmit coils that may be used to generate RF pulses to induce an oscillating magnetic field B₁. The transmit coil(s) may be configured to generate any type of suitable RF pulses.

The power management system 110 includes electronics to provide operating power to one or more components of the MRI system 100. In a non-limiting example, the power management system 110 may include one or more power supplies, energy storage devices, gradient power components, transmit coil components, or any other suitable power electronics needed to provide suitable operating power to energize and operate components of MRI system 100. As illustrated in FIG. 1, the power management system 110 may include a power supply system 112, power component(s) 114, transmit/receive circuitry 116, and may optionally include thermal management components 118 (e.g., cryogenic cooling equipment for superconducting magnets, water cooling equipment for electromagnets).

The power supply system 112 may include electronics that provide operating power to magnetic components 120 of the MRI system 100. The electronics of the power supply system 112 may provide, in a non-limiting example, operating power to one or more gradient coils (e.g., gradient coils 128) to generate one or more gradient magnetic fields to provide spatial encoding of the MR signals. Additionally, the electronics of the power supply system 112 may provide operating power to one or more RF coils (e.g., RF transmit and receive coils 126) to generate or receive one or more RF signals from the subject. In a non-limiting example, the power supply system 112 may include a power supply configured to provide power from mains electricity to the MRI system or an energy storage device. The power supply may, in some embodiments, be an AC-to-DC power supply that converts AC power from mains electricity into DC power for use by the MRI system. The energy storage device may, in some embodiments, be any one of a battery, a capacitor, an ultracapacitor, a flywheel, or any other suitable energy storage apparatus that may bi-directionally receive (e.g., store) power from mains electricity and supply power to the MRI system. Additionally, the power supply system 112 may include additional power electronics including, but not limited to, power converters, switches, buses, drivers, and any other suitable electronics for supplying the MRI system with power.

The amplifiers(s) 114 may include one or more RF receive (Rx) pre-amplifiers that amplify MR signals detected by one or more RF receive coils (e.g., coils 126), one or more RF transmit (Tx) power components configured to provide power to one or more RF transmit coils (e.g., coils 126), one or more gradient power components configured to provide power to one or more gradient coils (e.g., gradient coils 128), and may provide power to one or more shim power components configured to provide power to one or more shims (e.g., shims 124). In some implementations, the shims 124 may be implemented using permanent magnets, electromagnetics (e.g., a coil), or combinations thereof. The transmit/receive circuitry 116 may be used to select whether RF transmit coils or RF receive coils are being operated.

As illustrated in FIG. 1, the MRI system 100 may include the controller 106 (also referred to as a console), which may include control electronics to send instructions to and receive information from power management system 110. The controller 106 may be configured to implement one or more pulse sequences, which are used to determine the instructions sent to power management system 110 to operate the magnetic components 120 in a desired sequence (e.g., parameters for operating the RF transmit and receive coils 126, parameters for operating gradient coils 128, etc.). Additionally, the controller 106 may execute processes to estimate navigator maps for DWI reconstruction according to various techniques described herein. A pulse sequence may generally describe the order and timing in which the RF transmit and receive coils 126 and the gradient coils 128 operate to acquire resulting MR data. In a non-limiting example, a pulse sequence may indicate an order and duration of transmit pulses, gradient pulses, and acquisition times during which the receive coils acquire MR data.

A pulse sequence may be organized into a series of periods. In a non-limiting example, a pulse sequence may include a pre-programmed number of pulse repetition periods, and applying a pulse sequence may include operating the MRI system in accordance with parameters of the pulse sequence for the pre-programmed number of pulse repetition periods. In each period, the pulse sequence may include parameters for generating RF pulses (e.g., parameters identifying transmit duration, waveform, amplitude, phase, etc.), parameters for generating gradient fields (e.g., parameters identifying transmit duration, waveform, amplitude, phase, etc.), timing parameters governing when RF or gradient pulses are generated or when the receive coil(s) are configured to detect MR signals generated by the subject, among other functionality. In some embodiments, a pulse sequence may include parameters specifying one or more navigator RF pulses, as described herein.

Examples of pulse sequences include zero echo time (ZTE) pulse sequences, balance steady-state free precession (bSSFP) pulse sequences, gradient echo pulse sequences, inversion recovery pulse sequences, DWI pulse sequences, spin echo pulse sequences including conventional spin echo (CSE) pulse sequences, multi-shot FSE pulse sequences, turbo spin echo (TSE) pulse sequences or any multi-spin echo pulse sequences such a diffusion weighted spin echo pulse sequences, inversion recovery spin echo pulse sequences, arterial spin labeling pulse sequences, and Overhauser imaging pulse sequences, among others.

When capturing multi-shot FSE pulse sequences, the controller 106 can control the transit and receive coils 126 to generate a series of RF excitation pulses separated by intervals of time, during which the controller 106 controls the gradient coils 128 to apply phase-encoding gradients. The total number of phase-encoding steps may be divided into smaller shots, with each shot corresponding to a portion of the k-space. Each shot may be acquired with a separate RF excitation pulse, and the k-space data for each shot may be acquired and stored by the controller 106. The controller 106 can then combine the k-space data from all the shots to reconstruct the final image.

The controller 106 may initiate an example multi-shot FSE imaging process by first generating a strong magnetic field using the B₀ magnets 122. The strong magnetic field may align the hydrogen protons in the tissue. In some implementations, the B₀ magnets 122 can be permanent magnets that are always active. In some implementations, such as when the B₀ magnets 122 are electromagnets, the controller 106 may activate the B₀ magnets 122. The controller 106 can then initiate an RF pulse via transmit and receive coils 126 to excite the protons. The controller 106 can activate transmit and receive coils 126 to generate a series of RF refocusing pulses that are applied to the tissue, resulting multiple corresponding echoes. The phase of the RF pulses and the phase may be modified according to the techniques described herein.

After each excitation caused by the RF refocusing pulses, the controller 106 can utilize the gradient coils 128 to apply a phase-encoding gradient to the tissue being imaged. The phase of the phase-encoding gradient may be varied simultaneously with the varied phase of the RF excitation pulses. The controller 106 can vary the amplitude of the phase-encoding gradient for each excitation, allowing spatial information to be encoded along the phase-encoding direction. The controller 106 can then acquire the k-space data for each shot separately. As described herein, each shot can correspond to a portion of the k-space. The total number of shots depends on the number of phase-encoding steps utilized for the desired spatial resolution. The k-space data for the image can be stored in memory of the controller, and in some implementations, provided to the computing device 104. The controller 106 can then perform an image reconstruction process using the k-space data to generate an image-domain image of the scanned tissue. Although the examples herein provide features of the FSE imaging process, other types of imaging techniques for the MRI system can be utilized to generate images of the tissue of the subject associated with at least one body part.

The MRI system 100 may include one or more external sensors 178. The one or more external sensors may assist in detecting one or more error sources (e.g., motion, noise) which degrade image quality. The controller 106 may be configured to receive information from the one or more external sensors 178. In some embodiments, the controller 106 of the MRI system 100 may be configured to control operations of the one or more external sensors 178, as well as collect information from the one or more external sensors 178. The data collected from the one or more external sensors 178 may be stored in a suitable computer memory and may be utilized to assist with various processing operations of the MRI system 100.

The MRI system 100 may be a portable MRI system, and therefore may include portable subsystems 150. The portable subsystems 150 may include at least one power subsystem 152 and at least one motorized transport system 154. The power subsystem 152 may include any device or system that enables or supports the portability of the MRI system 100. In a non-limiting example, the power subsystem 152 may include any of the functionality of the power supply 112, and may further include other circuitry enabling the provision of electric power, including but not limited to as batteries and associated circuitry, AC-DC converters, DC-DC converters, switching power converters, voltage regulators, or battery charging circuitry, among others. The power subsystem 152 may include connectors that support the portability of the MRI system 100, such as connectors and cables of a suitable size for a portable system. In some implementations, the power subsystem 152 may include circuitry that provides power to the MRI system 100. In some implementations, the power subsystem 152 may include circuitry or connectors that enable the MRI system 100 to receive power from one or more power outlets, which may include standard power outlets.

The motorized transport system 154 can include any device or system that allows the MRI system 100 to be transported to different locations. The motorized transport system 154 may include one or more components configured to facilitate movement of the MRI system 100 to a location at which MRI is needed. In some implementations, the motorized transport system 154 may include a motor coupled to drive wheels. In such implementations, the motorized transport system 154 may provide motorized assistance in transporting MRI system 100 to one or more locations. The motorized transport system 154 may include a plurality of castors to assist with support and stability as well as facilitating transport.

In some implementations, the motorized transport system 154 includes motorized assistance controlled using a controller (e.g., a joystick or other controller that can be manipulated by a person) to guide the portable MRI system during transportation to desired locations. The motorized transport system 154 may include power assist circuitry (e.g., including accelerometers and vibration sensors, etc.) that detects when force is applied to the MRI system and, in response, engages the motorized transport system 154 to provide motorized assistance in the direction of the detected force. In some implementations, the motorized transport system 154 can detect when force is applied to one or more portions of the MRI system 100 (e.g., by an operator pushing on or applying force to a rail, housing, etc., of the MRI system 100) and, in response, provide motorized assistance to drive the wheels in the direction of the applied force. The MRI system 100 can therefore be guided to locations where MRI is needed. The power subsystem 152 can be utilized to provide power to the MRI system 100, including the motorized transport system 154.

In some implementations, the motorized transport system 154 may include a safety mechanism that detects collisions. In a non-limiting example, the motorized transport system 154 can include one or more sensors that detect the force of contact with another object (e.g., a wall, bed or other structure). Upon detection of a collision, the motorized transport system 154 can generate a signal to one or more motors or actuators of the motorized transport system 154, to cause a motorized locomotion response away from the source of the collision. In some implementations, the MRI system 100 may be transported by having personnel move the system to desired locations using manual force. In such implementations, the motorized transport system 154 may include wheels, bearings, or other mechanical devices that enable the MRI system 100 to be repositioned using manual force.

As illustrated in FIG. 1, the computing device 104 may communicate with the controller 106, for instance, receive the MR data. The computing device 104 can be configured to process the MR data from the controller 106. In a non-limiting example, the computing device 104 may process received MR data to generate one or more MR images using any suitable image reconstruction processes. Additionally or alternatively, the controller 106 may process received MR data to perform image reconstruction, and the reconstructed image may be provided to the computing device 104, such as for display. The controller 106 may provide information about one or more pulse sequences to computing device 104 for the processing of data by the computing device.

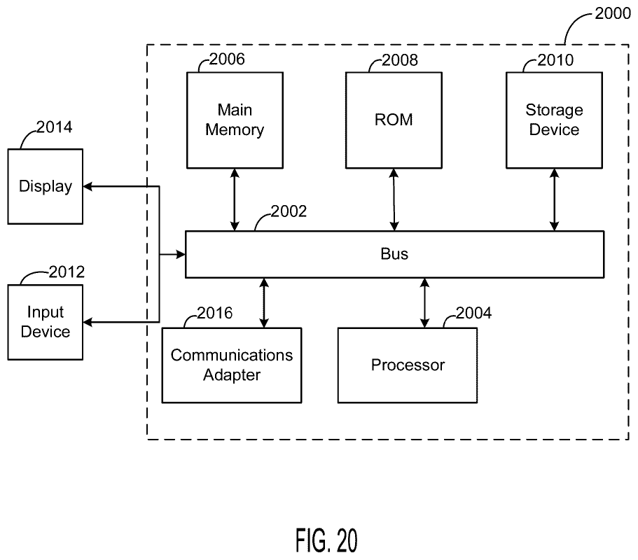

The computing device 104 may be any electronic device configured to process acquired MR data and generate one or more images of a subject being imaged. The computing device 104 may include at least one processor and a memory (e.g., a processing circuit). The memory may store processor-executable instructions that, when executed by a processor, cause the processor to perform one or more of the operations described herein. The processor may include a microprocessor, an application-specific integrated circuit (ASIC), a field-programmable gate array (FPGA), a graphics processing unit (GPU), a tensor processing unity (TPU), etc., or combinations thereof. The memory may include, but is not limited to, electronic, optical, magnetic, or any other storage or transmission device capable of providing the processor with program instructions. The memory may further include a floppy disk, CD-ROM, DVD, magnetic disk, memory chip, ASIC, FPGA, read-only memory (ROM), random-access memory (RAM), electrically erasable programmable ROM (EEPROM), erasable programmable ROM (EPROM), flash memory, optical media, or any other suitable memory from which the processor may read instructions. The instructions may include code generated from any suitable computer programming language. The computing device 104 may include any or all of the components and perform any or all of the functions of the computing system 2000 described in connection with FIG. 20. In some implementations, the computing device 104 may be located in a same room as the MRI system 100 or coupled to the MRI system 100 via wired or wireless connection. In some implementations, the computing device 104 can be remote from the MRI system 100 and/or the controller 106 (e.g., in a different location), configured to receive data or information via a network.

In some implementations, computing device 104 may be a fixed electronic device such as a desktop computer, a server, a rack-mounted computer, or any other suitable fixed electronic device that may be configured to process MR data and generate one or more images from MR signals captured using the MRI system 100. Alternatively, computing device 104 may be a portable device such as a smart phone, a personal digital assistant, a laptop computer, a tablet computer, or any other portable device that may be configured to process MR signal data. In some implementations, computing device 104 may comprise multiple computing devices of any suitable type, as aspects of the disclosure provided herein are not limited in this respect. In some implementations, operations that are described as being performed by the computing device 104 may instead be performed by the controller 106, or vice-versa. In some implementations, certain operations may be performed by both the controller 106 and the computing device 104 via communications between said devices. For purposes of examples herein, the computing device 104 can execute instructions to simulate structures or images for improving model performance.

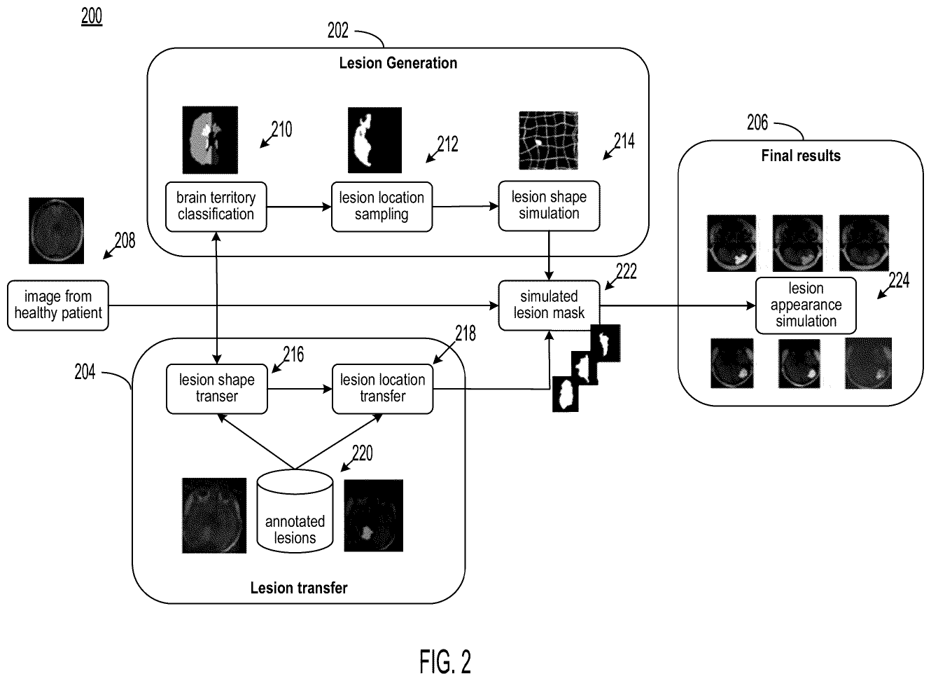

FIG. 2 depicts an example pipeline 200 for simulating structures for at least one image, in accordance with one or more implementations. As described herein, the computing device 104 (or in some cases, the controller 106) can be configured to simulate various structures or images, for instance, by performing the features or operations of the example pipeline 200. The example pipeline can include various operations to be executed by the computing device 104 for structure or image simulation, such as but not limited to at least operations 208-224. One or more of these operations can be a part of at least one of a lesion generation stage 202, a lesion transfer stage 204, and/or a result stage 206. The example pipeline 200 can include other operations or stages to perform the techniques described herein.

As an initial step, at operation 208, the computing device 104 can receive/obtain/acquire at least one image of a subject/patient. For purposes of examples for simulating a lesion, the image can be from a healthy subject (e.g., the image of healthy tissues or structures), although images with abnormalities may be utilized in some other cases. The computing device 104 can obtain the image from the controller 106. In some cases, the computing device 104 can retrieve the image from a data repository, such as a database on a server or a storage device of the controller 106. The computing device 104 can use the image can show at least one body part of the subject, such as the brain. The image can be a 2D image or a 3D image. The image may include a layer or slice of the body part, such as a 2D slice of the 3D image.

In some aspects, the computing device 104 can use the image for generating a lesion (e.g., in the lesion generation stage 202). In some aspects, the computing device 104 can use the image for lesion transfer (e.g., in the lesion transfer stage 204). In various aspects, the computing device 104 can utilize the image for a simulated mask (e.g., lesion mask), for instance, to apply or embed the simulated lesion mask on the image. The image can be processed sequentially and/or concurrently at various stages or operations of the example pipeline 200. In some cases, when applying the mask to the image (e.g., an input image or a first image), the computing device 104 can generate another image (e.g., an image with the simulated lesion, sometimes referred to as an output image or a second image) as part of the result stage 206. The output image can include a similar appearance to the input image, with the applied simulated lesion at a location on the input image, for example. For purposes of examples, the operations of the example pipeline 200 can be used for simulating at least one lesion (e.g., abnormality) in the brain. In some other aspects, the operations of the example pipeline 200 may be used for simulating other structures, such as but not limited to healthy tissues, in other parts of the body, not limited to the brain.

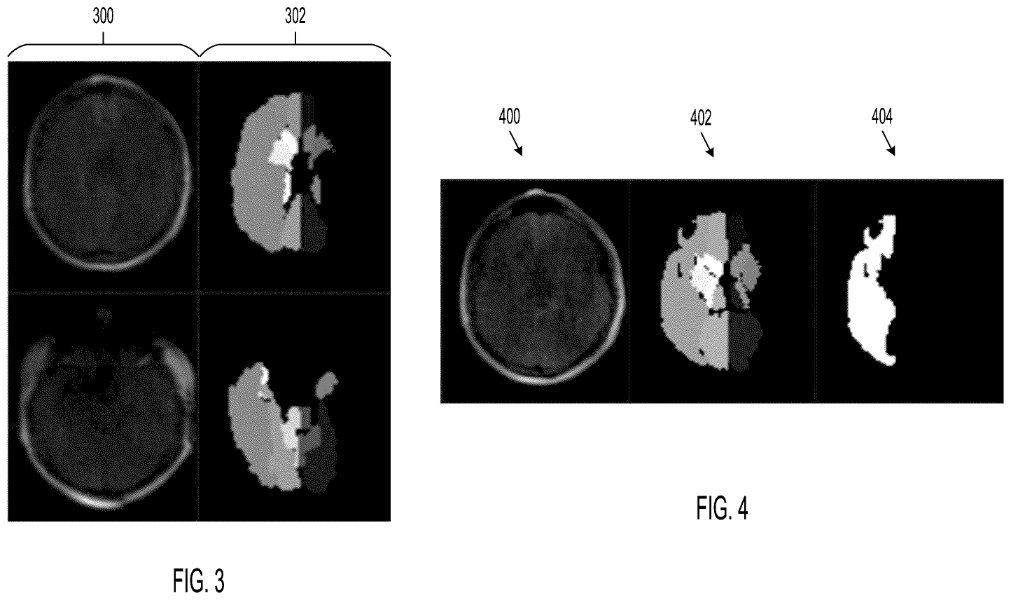

In the lesion generation stage 202, the computing device 104 can perform operations 210-214 for generating/creating an example lesion (e.g., simulated lesion mask). This lesion mask can be artificially created by the computing device 104. At operation 210, the computing device 104 can perform territory classification for at least one anatomical region associated with the body part (e.g., at least a portion of the body part). The operation 210 can be described in conjunction with at least one of, but not limited to, FIGS. 3-4. In various configurations, the computing device 104 can perform at least one or a combination of the following example methods/approaches/techniques for lesion generation:
Brain anatomical region or brain vascular territory (or anatomical region of other body parts);
Region-growing approach; and/or
Data-driven pathology mask generation.

To generate the example lesion using brain territory anatomy, the computing device 104 can identify the territories associated with the body part. FIG. 3 depicts an example of brain territory classification, in accordance with one or more implementations. As shown, FIG. 3 includes example images 300 or slices (e.g., 2D slices) of the brain, e.g., from the input image. In some cases, the images 300 can be multiple slices of the brain in 3D. The computing device 104 can process each image 300 to separate/divide or extract a number of vessel territories (e.g., 26 vessel territories) associated with the image 300, e.g., extract territories from the brain. The computing device 104 can utilize at least one suitable territory classification or mapping technique to separate the territories of the body part, such as at least one of anatomical landmarks, voxel-based (or pixel-based) morphometry (VBM), parcellation algorithms, diffusion tensor imaging (DTI), and/or functional connectivity analysis, among others. For example, the territories may include but are not limited to major left and right vessel branches, such as the middle cerebral artery (MCA), anterior cerebral artery (ACA), posterior cerebral artery (PCA), anterior inferior cerebellar artery (AICA), posterior inferior cerebellar artery (PICA), superior cerebellar artery (SCA), vascular territories, etc. One or more territories can be a part of the at least one anatomical region.

According to these separated territories, the computing device 104 can generate or obtain a territory map of each image 300. The territory map can be included as part of each image 302 associated with the respective image 300, where each contrasting gray scale portion of the images 302 can represent a respective territory (e.g., unique vessel territory) associated with the body part. The generated images 302 can be in 2D or 3D.

At operation 212, the computing device 104 can identify or select a location (e.g., lesion territory) for sampling. FIG. 4 depicts an example of at least one territory selected as a targeted location of a simulated lesion, in accordance with one or more implementations. For example, the computing device 104 can obtain image 400 for processing. The computing device 104 can separate various territories from the image 400 to generate image 402 including a mapping of the territories. Responsive to obtaining or detecting the territories, the computing device 104 can select at least one of the territories as a targeted lesion region (e.g., a region to apply a simulated lesion). For instance, the computing device 104 can sample at least one territory uniformly or with customized or predetermined multinomial distribution. Sampling the territory uniformly can refer to selecting elements or data points from the territory such that each element has a similar probability of being included as part of the selected sample, in this case, the location. Sampling the territory with the customized multinomial distribution can involve selecting elements or features from the territory using a distribution configuration tailored to certain characteristics or proportions of the elements within the territory, thereby allowing certain elements of the territory to be more or less apparent as part of the selected territory. In some cases, the computing device 104 may use the customized multinomial distribution for territory selection if certain territories are preferred or desired over other territories, for example. As shown in FIG. 4, responsive to sampling the at least one territory, the computing device 104 can select an associated region (e.g., the location) for the targeted lesion. An example region (or territory) selected as the location for the targeted lesion can be shown in the example image 404 of FIG. 4.

In some implementations, the computing device 104 may be configured to select individual territories (or regions) for targeting at least one lesion. For example, during a first cycle of executing the operations of the example pipeline 200, the computing device 104 may select a first territory for applying a first simulated lesion. In a second cycle, the computing device 104 may select a second territory for applying a second simulated lesion, and so forth.

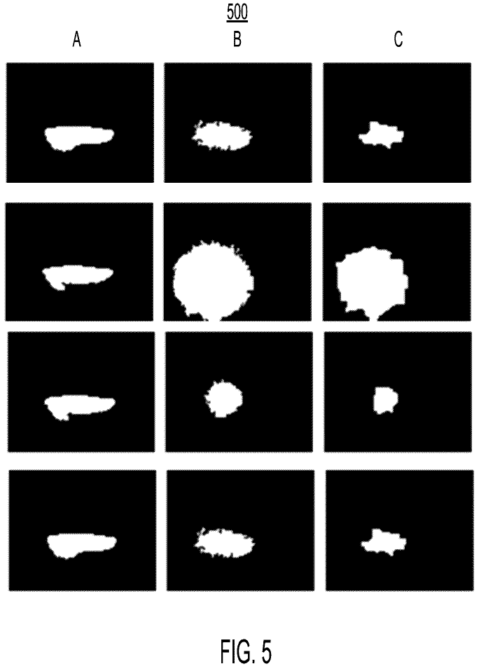

At operation 214, responsive to selecting or identifying the location, the computing device 104 can simulate the shape (e.g., including size) of the example lesion (e.g., for lesion generation). FIG. 5 depicts an example 500 of lesion masks generated with different shapes and sizes, in accordance with one or more implementations. To create variations in the shape and size of the example lesion, the computing device 104 can generate a shape (e.g., 2D shape) controlled by or according to one or more parameters, such as a long axis and/or a short axis. The long axis can refer to the longest dimension or alignment of the structure or lesion, in this case. The short axis can refer to a cross-sectional view of the structure that is perpendicular to the long axis. The one or more parameters may define the size of the example lesion.

The shape may be elliptical, circular, jagged, annular, crescentic, etc. The computing device 104 may select the shape based on an indication or a configuration by the user 102. In some cases, the computing device 104 may select the shape from a list of shapes, using any suitable selection technique, such as random selection, sequential selection (e.g., for multiple simulations of lesion masks), weighted selection, etc.

For example, the size of the example lesion may be within the one or more parameters, which may indicate the maximum (or the minimum) dimension of the example lesion. The one or more parameters may be based on the selected location (e.g., the sampled territory) to which to apply the example lesion. For instance, the computing device 104 can determine the dimension, including the long axis and/or the short axis, associated with the selected region or location of the body part. These long axis and/or short axis can be used to define the size of the example lesion, such that the size of the example lesion does not exceed the size of the selected lesion, for example. In some cases, the long axis and/or the short axis can be configured by the user 102.

Subsequently, the computing device 104 can use the selected or determined shape and size, at least in part, to simulate the example lesion (e.g., simulating lesion mask), such as at operation 222. For example, column A (e.g., the first column) of example 500 can provide example images of lesion masks from the same territory having different shapes and/or sizes. In various configurations, with the generated shape and size, such as in column A of the example 500, the computing device 104 can apply at least one type of distortion for creating variations to the shape and size of the example lesion. The computing device 104 can apply the at least one type of distortion using at least one suitable image warping or image deformation technique, such as grid-based deformation, thin-plate splines, free-form deformation, etc. The type of distortion can include but is not limited to at least one of elastic distortion, elastic deformation, field inhomogeneity, ghosting artifacts, slice profile artifacts, and/or magnetic-induced distortion, among others. Examples of the distortion (e.g., elastic distortion) applied to the shape and size can be shown in at least example images of columns B-C of the example 500. By applying the distortion, the shape and size of the example lesion can be further modified/changed.

In some configurations, to simulate the lesion mask using the region-growing approach/technique/method, the computing device 104 can create/generate or simulate the lesion mask by using at least one suitable region-growing technique, such as watershed, seed-filling, region merging, etc. By utilizing the region-growing approach, the computing device 104 can maintain/keep the lesion within an anatomically consistent region (e.g., within the same territory or associated territories of the body part) by isolating (or grouping) regions/locations of the body part with similar intensities. The intensities can be determined based on the brain territory classification, at operation 210, for example.

For example, the computing device 104 can initialize or place at least one seed within the input image (e.g., brain MRI image) at a desired location (e.g., such as the selected location at operation 212). The seed can correspond to a starting point for lesion growth (e.g., growing the lesion mask). In some cases, the seed can represent the location from which the abnormality starts to grow. The number of seeds can be adjusted/updated according to the desired number of lesion locations/areas to be grown. In some cases, the seed can be represented by an intensity or a contrast of a pixel or voxel within the image, for example.

Responsive to placing the seed, the computing device 104 can iteratively grow the region around the seed (e.g., starting the region-growing process). If there are more than one seed, the computing device 104 can iteratively grow multiple regions associated with the respective seeds. Growing the region around the seed can include extending the region to voxels neighboring or adjacent to the seed, among other previously grown regions. The region can extend to neighboring voxels having similar intensity (e.g., voxel intensity) as the voxel where the seed is placed. By growing the region to voxels having the similar intensity, the computing device 104 can ensure that the example lesion for simulating the lesion mask is within the at least one selected location or territory. The number of iterations for growing the region can be predetermined or configured by the user 102.

In some implementations, the number of iterations can be based on at least one of the selected territory, such as the type of anatomical territory or the size of the territory, or the predetermined parameters, such as the long axis and/or short axis. For example, the computing device 104 can iteratively grow the region around the seed until the dimension of the extended region is at or around a certain percentage of the dimension of the territory, such as 10%, 20%, or 30%. In another example, based on the type of anatomical territory selected, the computing device 104 can determine an associated dimension (e.g., maximum size) or the number of iterations to extend the region around the seed. In yet another example, the computing device 104 may continue to grow the neighboring region until satisfying the long axis and/or short axis parameters. Specifying or configuring the number of iterations can allow control over the final dimension of the lesion mask.

The computing device 104 can apply the at least one suitable region-growing technique to separate the growing regions (e.g., locations of the body part for growing regions) at boundaries where the change in intensity is at or above a threshold. The threshold can be predetermined by the user 102. The change in intensity can be compared between a first intensity at the seed location or at the extended region around the seed and a second intensity at a potential location for growing the region. For example, the intensity may be represented by a value ranging from 0 to 1, where 0 can represent the lowest intensity and 1 can represent the highest intensity, or vice versa. If the threshold is set as 0.2 and the intensity at the seed is 0.5, the computing device 104 can separate the growing regions at the boundaries with intensity above 0.7 or below 0.3, for example.

By detecting the changes in the intensity, the computing device 104 can prevent the growing region (e.g., as part of the simulated lesion mask) from spreading into areas of dissimilar intensities, thereby maintaining the region within the same anatomical region or territory. Responsive to iteratively growing the region, the computing device 104 can generate a lesion mask (e.g., binary lesion mask), such as at operation 222, based on the grown regions around the seed. The lesion mask can simulate a lesion that is grown at, around, or within the body part while adhering/conforming to the anatomical boundaries according to the intensities of the image.

In some configurations, to simulate the lesion mask using the data-driven pathology mask generation, the computing device 104 may use historical (e.g., existing) lesion data to generate the lesion mask. In this case, the computing device 104 can leverage or utilize at least one suitable ML technique, such as generative adversarial networks (GANs), variational autoencoders (VAEs), autoregressive models, deep Boltzmann machines (DBMs), etc., and database with historical images. These historical images may include brain images with and/or without abnormalities (e.g., lesions) depending on the type of structures to simulate, for instance, healthy tissues or lesions. Utilizing the techniques discussed herein for generating the lesion mask can provide a realistic representation of the structure of the body part, such as healthy brain tissues or brain lesions, among others.

For example, the computing device 104 can train a model using the at least one suitable machine learning technique with the historical images of lesions. The computing device 104 can feed the historical images as training data (or input data) for the model. The model, using the machine learning technique, can learn various features, patterns, and/or characteristics associated with the various structures of the body part, such as the shape, size, or other details associated with the lesions. The model can be composed of or include a generator configured to generate/create at least one lesion mask according to the trained data. The generated lesion mask can imitate or simulate the real mask associated with the training data, such as having similar or comparable shapes, sizes, and/or other details to real lesions, for example. In some cases, the model may execute at least one other machine learning technique for tuning certain features of the simulated lesion mask.

Further, the model can be composed of or include a discriminator configured to evaluate the fidelity associated with the generated lesion mask. To perform the evaluation, the model can be configured with metrics or evaluation criteria, such as structural similarity index (SSIM), mean squared error (MSE), or peak signal-to-noise ratio (PSNR) associated with the lesions. The model may preprocess and/or normalize the data by configuring or defining the data range, removing outliers, and/or applying suitable transformation techniques to ensure the data is in a comparable format, for instance, between the actual/real lesion and the simulated example lesion. The model can perform at least one of visual inspection, statistical analysis, quantitative measurement, or other operations (not limited to those herein) for identifying similarities and/or differences between the simulated example lesion and the real lesions (or real mask).

In some implementations, the computing device 104 can execute the model for refining the simulated lesion mask. For example, the model can apply at least one post-processing technique or operation to improve the quality, anatomical consistency, etc., of the generated lesion masks. The post-processing technique for fidelity improvement may include but is not limited to at least one of texture mapping, post-processing filter, lighting and shading configuration, noise addition, or in some cases, motion blur, among others. The post-processing techniques can enhance the realism of the simulated lesion mask, for instance, by introducing characteristics (e.g., imperfections) produced by the real-world data, such as when capturing images via the imaging device, movements, or lighting effects.

In some implementations, such as in the lesion transfer stage 204, the computing device 104 can transfer one or more historical lesions to the input image. In some implementations, the simulated lesion mask from the lesion transfer stage 204 can be used independently of the simulated lesion mask from the lesion generation stage 202. In some other implementations, the simulated lesion mask from the lesion transfer stage 204 can be used in combination with the simulated lesion mask from the lesion generation stage 202, such as including multiple lesion masks from both the lesion generation stage 202 and the lesion transfer stage 204.

To transfer the lesion, at operation 220, the computing device 104 can obtain one or more historical images with annotated structures (e.g., annotated lesions and/or healthy tissues) from a data repository. The data repository can be local to or remote from the computing device 104. The annotated structures can refer to data embedded or associated with the respective historical image, which can indicate various details associated with the lesions of presented in the historical image. For example, the computing device 104 can retrieve an annotation of at least one of the shapes, sizes, locations, colors, etc., associated with the lesions.

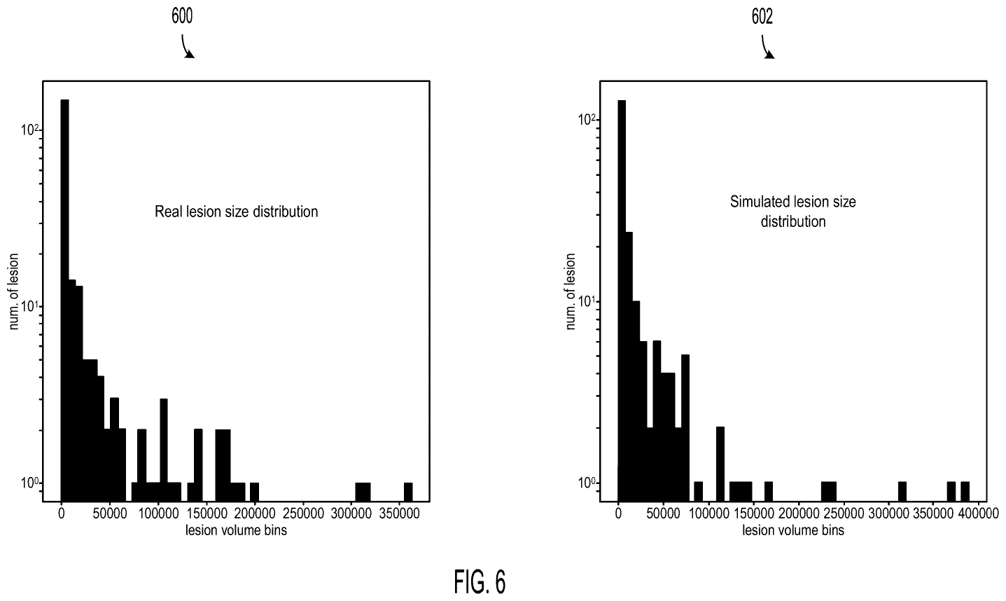

At operation 216, the computing device 104 can transfer or extract the lesion shape from the historical image. At operation 218, the computing device 104 can transfer or extract the location of the lesion from the historical image. By using the lesion from the annotations from the historical images, the lesion can be relatively similar to or closely match the real lesions (e.g., real shapes, size distribution, etc.). In some cases, the computing device 104 may transfer the lesion shape, size, and/or location, among other details from the historical images to the input image as part of a simulated mask, such as at operation 222. For comparison between the volumes of the real lesions and the simulated lesions from the lesion transfer stage 204, FIG. 6 depicts example graphs 600, 602 for volume distributions of real lesions and simulated lesions with lesion transfer, in accordance with one or more implementations. For example, the graph 600 can illustrate the size or volume distribution for real lesions from historical images. The graph 602 can illustrate the size or volume distribution for the simulated lesions with the lesion transfer operation. By comparison, for example, the volume distributions between real lesions and simulated lesions can be comparable, because the simulation is based on the annotated information of real lesions.

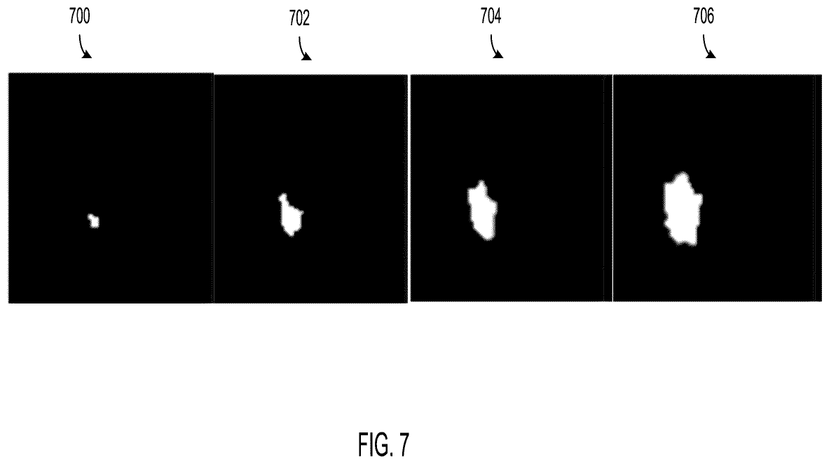

In some configurations, at or as part of the operation 222, the initial mask (e.g., lesion mask or normal mask) simulated by the computing device 104 may be in 2D. In this case, the computing device 104 can extend or expand the 2D simulated mask to a 3D simulated mask. FIG. 7 depicts an example of 2D slices 700-706 (e.g., 2D simulated masks) including a generated lesion for interpolation, in accordance with one or more implementations. For example, for a lesion generation path to extend the 2D simulated mask, the computing device 104 can interpolate the 2D lesion mask across slices in 3D, such as to extend the 2D mask across multiple adjacent slices in a 3D volume of the image.

As shown in FIG. 7, at least one of the 2D slices 700-706 can correspond to a key slice (e.g., the first 2D simulated mask from the lesion generation stage 202 or the lesion transfer stage 204. The computing device 104 can interpolate to at least one side (e.g., at least one of left, right, top, and/or bottom) of the key slice to generate the example 2D slices 700-706 of FIG. 7. The at least one side can be configurable by the user 102 or according to the key slice. For example, if the key slice is simulated from the left side of the lesion, the computing device 104 can interpolate from left to right to generate the 3D simulated mask. If the key slice is simulated from the right side of the lesion, the computing device 104 can interpolate from right to left to generate the 3D simulated mask, and so forth. In such cases, the 2D slices 700-706 of FIG. 7 may represent individual layers of at least a portion of the 3D simulated mask. For purposes of providing examples, the 2D slices 700-706 can represent an example of 2D slices interpolated from left to right, respectively, to form at least a portion of a 3D lesion.

In some implementations, for simulated lesion mask from the lesion transfer stage 204, the computing device 104 may simulate a 3D mask by transferring the shape, size, and/or location of the historical lesions. In such cases, the computing device 104 may not be required to perform the 2D simulated mask for extending the lesion mask from the lesion transfer stage 204.

Responsive to simulating the lesion mask, the computing device 104 can proceed to the result stage 206. At operation 224 of the result stage 206, the computing device 104 can simulate the appearance of the simulated lesion mask. For example, the lesion mask from the operation 222 can indicate the location and/or the shape of the simulated lesion. With the simulated lesion, the computing device 104 can determine the appearance of the lesion, including but not limited to pixel or voxel intensities for the simulated lesion. To maximize or optimize the fidelity in the simulated lesion mask, the computing device 104 may perform at least one of a feature-engineering configuration (or approach) or data-driven configuration for simulating the appearance of the lesion, as described herein. The appearance of the lesion can be a part of the lesion mask, for example.

### Example Feature-Engineering Configuration

In various configurations, as part of the feature-engineering configuration for simulating the appearance, the computing device 104 can select an average (or mean, median, mode, etc.) pixel or voxel intensity of the lesion for simulating the lesion appearance. For example, depending on the application context (e.g., type of abnormalities of the body part), the location (or anatomical region) associated with the lesion, etc., the lesion may be at least one or a combination of hyper-intense, hypo-intense, iso-intense, etc. The computing device 104 can determine or select the average intensity according to or based on the type of abnormality to simulate for the lesion mask. Each type of abnormality for a particular body part may be stored in a look-up table or in association with their respective average intensity for various images. For instance, a white matter disease lesion on fluid-attenuated inversion recovery (FLAIR) may be presented as hyper-intense in images, and a stroke lesion on apparent diffusion coefficient (ADC) may be presented as hypo-intense, in some cases.

Because different abnormalities may be associated with varying intensities (e.g., which may depend on the intensity of other portions of the image), the computing device 104 can utilize the techniques or mechanisms discussed herein to provide customization on the level of intensity for the simulated lesion. For example, the computing device 104 can account for the intensity distribution (e.g., averages, medians, modes, etc.) of the 3D series of the image. The 3D series can include consecutive 2D images or slices constructed to form the 3D image. The intensity distribution can be separated/divided into multiple intervals or categories, such as the following example:
very_low: (min, low_1_percentile)
low: (low_1_percentile, low_2_percentile)
high: (top_2_percentile, top_1_percentile)
very_high: (top_1_percentile, volume_max)

The intensity distribution may be separated into one or more sources (e.g., one or more intervals, a set of values, or in some cases overlapping intervals). In some implementations, the intensity associated with the simulated lesion mask may be sampled (e.g., randomly sampled) from one or more of the intervals, such as with a predetermined (e.g., user-defined) probability configuration. For example, to simulate a stroke lesion in DWI, the computing device 104 can obtain a probability configuration including a sampling probability of p=(0, 0, 0.05, 0.95), such that there is a relatively higher chance/probability of giving a lesion high intensities relative to the remainder or other portions of the brain tissues. Depending on the selected probability configuration, the resulting stroke lesion intensity distribution for the appearance of the lesion can resemble the corresponding real lesions.

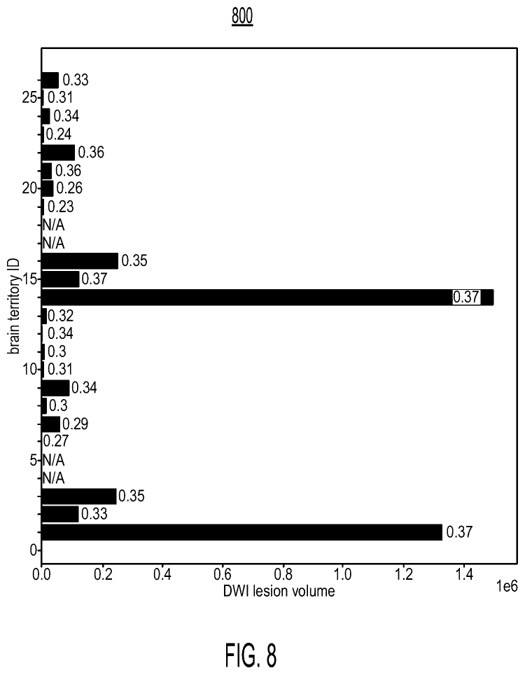

For example, FIG. 8 depicts an example bar graph 800 for the intensity distribution of lesions on DWI, in accordance with one or more implementations. The example bar graph 800 can be derived or constructed from real DWI data, for example. The range of the pixel or voxel intensity in the example bar graph 800 can be normalized to a range of 0 to 1. In various aspects, given a lesion mask, an intensity value can be assigned to the lesion mask for insertion into the input image. To determine the intensity value to assign, the computing device 104 can receive one or more intervals for sampling, where each interval can include a set of intensities (e.g., intensity values). With the sets of intensities for sampling, the sampling probability distribution (e.g., the resulting distribution) can be determined, such that when certain statistics are computed, similarly to the statistics shown in FIG. 8 from real DWI lesions to synthesized/simulated DWI lesions, the distribution (e.g., mean of intensity and volume) may be the same between the real and simulated DWI lesions. When the intensity distribution of the simulated lesion is similar to the intensity distribution of a real lesion, the computing device 104 can determine that the simulated lesion with the intensity value(s) has relatively high fidelity, and the respective set of intervals can be used for determining the intensity value. Otherwise, if the intensity distribution of the simulated lesion is dissimilar to the intensity distribution of the real lesion, the computing device 104 can determine that the simulated lesion with the intensity value(s) has relatively low fidelity. As shown, for real stroke lesions on DWI with the sampling probability of p=(0, 0, 0.05, 0.95), there may be substantially more DWI lesion volumes between territory IDs ranging from 0-5 and 13-17, compared to other territory IDs. The territory ID can represent a number from a range (e.g., 1 to 26, in this case) representing different anatomical regions, where each territory ID can be associated with a respective territory of the body part, such as 1 for the left lateral ventricle, 2 for the right lateral ventricle, etc.

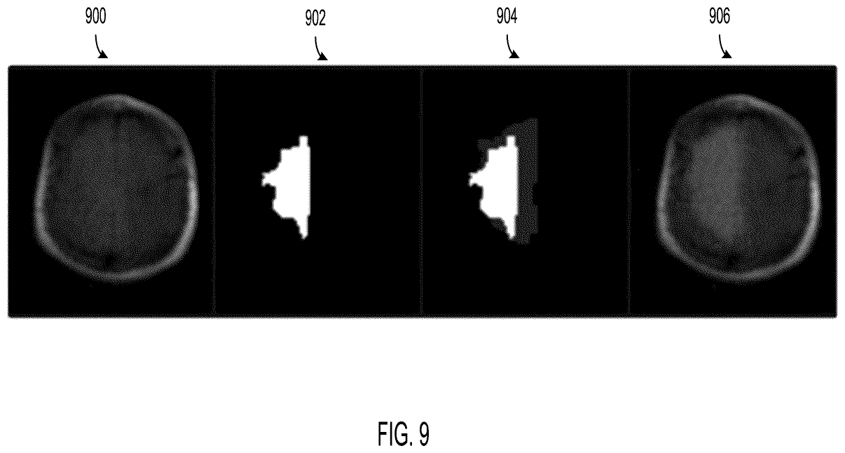

In some implementations, the computing device 104 can add one or more patterns (e.g., edema patterns) in the lesion for simulating the appearance. The computing device 104 can add the one or more patterns additionally or alternatively to augmenting or simulating the lesion intensity. For example, for certain types of abnormality, such as edema, the effect of the edema may be applied or added to the simulated example lesion as part of the appearance. The effect may be represented by the patterns. FIG. 9 depicts an example of a simulated edema effect around a lesion, in accordance with one or more implementations. As shown, the computing device 104 can simulate the lesion mask in example image 902 for at least one anatomical region or territory of the body part in example image 900. The computing device 104 can simulate the pattern of the edema, for instance, by creating a region with relatively lower intensity around at least a portion of the simulated lesion mask, as shown in example image 904. For instance, the computing device 104 may provide the pattern of the edema as a layer below the lesion mask or around the example lesion. The computing device 104 can adopt different levels of intensities from the lesion core, e.g., the additional simulated lesion, such as the example edema mask in FIG. 9, can include different intensity values compared to the first lesion applied/inserted in the image. Responsive to simulating the pattern as part of the appearance, the computing device 104 can apply the appearance to the lesion mask, which can be inserted or applied to the input image, for example, to generate the example image 906.

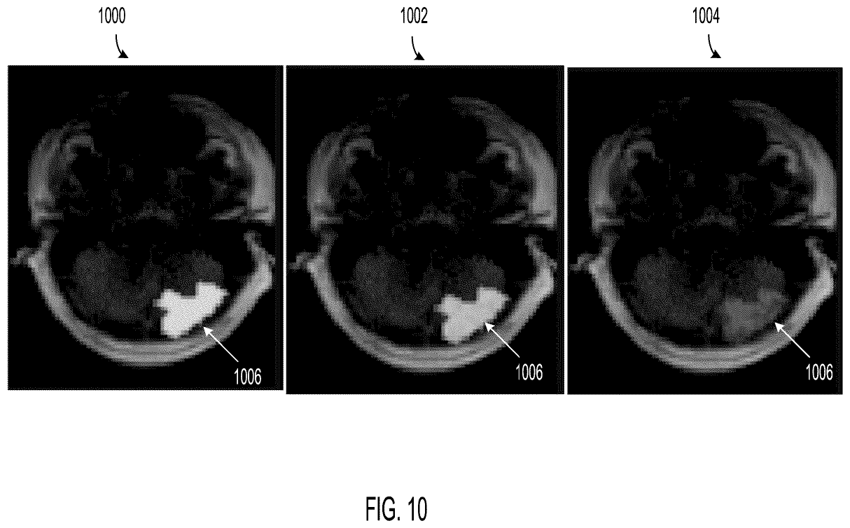

In some implementations, the computing device 104 may add or insert device acquisition noise as part of the appearance for the simulated lesion. FIG. 10 depicts example images 1000-1004 of acquisition noise in the simulated lesion, in accordance with one or more implementations. For example, depending on the type of imaging device (e.g., low-field MRI scanner, CT scanner, etc.) used to capture the images, environmental interference and/or other acquisition noises may be introduced in the images of the subject. Hence, the computing device 104 can simulate the appearance of the simulated lesion mask to have an inherent inhomogeneity (or certain types of noises) to improve the fidelity associated with the simulation.

To simulate the appearance of the acquisition noise, the computing device 104 can drop or remove pixels or voxels with a probability (e.g., labeled as “drop_prob”). By dropping certain pixels or voxels according to the probability, the simulated lesion may appear with relatively less intensity. The drop probability may be a range of 0 to 1, where 0 can represent no drop of pixels or voxels and 1 can represent that all pixels and voxels associated with the simulated lesion are to be dropped. Subsequently, the computing device 104 may apply a smoothing technique (e.g., Gaussian smoothing, among other techniques) (e.g., labeled as “sigma” for the configured value of the smoothing) on the simulated mask. By applying the smoothing technique, the computing device 104 can reduce the blur sharp transitions between pixels or voxels of the image. The computing device 104 may apply other processing techniques to imitate the acquisition noise of the imaging device or other noises potentially captured in the images. As shown in FIG. 10, example image 1000 can include a simulated lesion 1006 with a drop_prob of 0 (e.g., no pixel drop) and a sigma of 2 (e.g., two times more extensive blurring of the pixels, where respective 2 pixels in each direction are aggregated or blended to create smooth transitions). In some cases, a sigma of 0 can provide staircase-like boundaries between pixels (e.g., no smoothing between pixels). In some other cases, a relatively high sigma number, such as 20, can provide a relatively smooth transition (e.g., blending of pixel contrasts or intensities) between the corresponding number of pixels in various directions. In various aspects, the computing device 104 can create smooth transitions between healthy tissue and the inserted pathology regions. When increasing the drop probability, such as to 0.2 in example image 1002, the simulated lesion 1006 can be seen with relatively lower intensity compared to the example image 1000. When further increasing the drop probability to 0.8 in example image 1004, the simulated lesion 1006 can be seen with even lower overall intensity compared to the other example images 1000, 1002, thereby blending in relatively more naturally with other portions of the image (e.g., healthy tissues around the lesion). By utilizing the drop probability, the computing device 104 can simulate various different types of textures. For example, different types of abnormalities can include different textures. By using different drop probabilities, the computing device 104 can simulate various types of abnormalities for detection, such as but not limited to stroke, hemorrhage, tumor, etc.

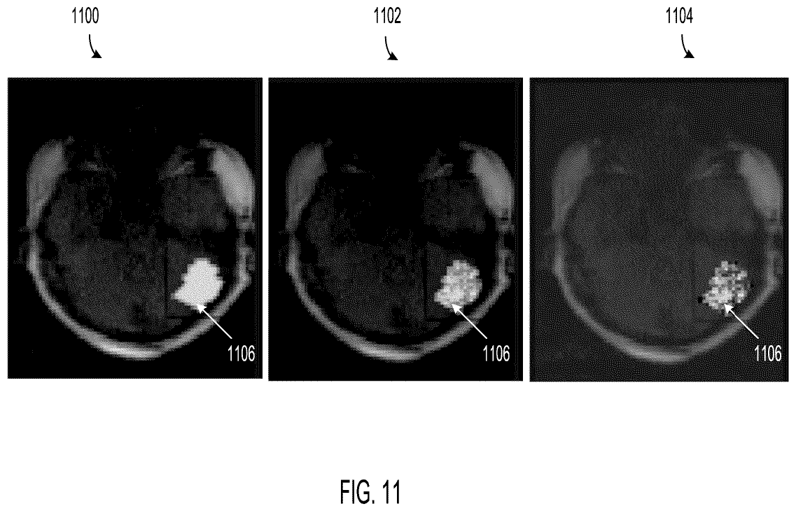

In some implementations, the computing device 104 can add Gaussian noise in the simulated lesion as part of the lesion appearance. FIG. 11 depicts an example of different noise levels for the image, such as different levels of Gaussian noises, in accordance with one or more implementations. For example, the computing device 104 can sample Gaussian noise (e.g., generate a value from a Gaussian distribution) for individual lesion mask pixels or voxels to create a noise signal. The value of the pixel or voxel can be multiplied or computed according to the constant (e.g., the value of the Gaussian noise) to attenuate or amplify the indication of the noise. Applying the Gaussian noise can produce/generate lesions with a relatively granular appearance (e.g., lesions that appears grainy). In this case, applying the Gaussian noise may decrease the smoothness of the simulated lesion mask (e.g., opposite to applying the dropout mask simulating the acquisition noise). The parameter of the Gaussian noise may be based on the maximum pixel value (e.g., pixel intensity or the brightness value of the pixel). For example, the Gaussian noise can be a range of [0, pixel_value_max]. The computing device 104 can enhance and/or attenuate the simulated lesion intensity, which can change the overall image contrast as it is scaled based on the minimal and maximal pixel values (or from the minimum to the maximum pixel values) after lesion insertion.

For example, as shown in example image 1100 of FIG. 11, the noise level can be zero for the simulated lesion mask. In this case, the simulated lesion 1106 may be affected by Gaussian noise with a base standard deviation, such as 1. The base standard deviation can be multiplied by a constant (e.g., 100, 250, etc.), which can refer to a noise level. The upper bound/limit of the constant can correspond to the pixel_value_max (e.g., highest pixel value in the image). The computing device 104 can receive a configuration from the user 102 or obtain a predetermined configuration indicating to increase the noise level to 100, as in example image 1102. Increasing the noise level (e.g., Gaussian noise level) can increase the variability or randomness in the pixel values, thereby resulting in reduced clarity, such as a less clear simulated lesion, reduced fine details, reduced signal-to-noise ratio (SNR), or in some cases the overall intensity value, among others. In this case, various pixels of the simulated lesion of the example image 1102 can be affected by the Gaussian noise. As shown, higher Gaussian noise can result in a decrease in the overall clarity (or in some cases intensity) of the simulated lesion. The computing device 104 can further increase the noise level to 250 as in example image 1004. As shown, the pixels associated with the simulated lesion (e.g., for noise level of 250 in this case) may be further affected by the Gaussian noise, hence, the overall clarity of the simulated lesion can be further decreased. The higher the noise level, the lower the overall intensity of the simulated lesion.

In some implementations, the computing device 104 can simulate a mass effect as part of the appearance of the simulated lesion. The mass effect can refer to how anatomical structures or regions of an image deform or distort due in response to the growth of the lesion or the type of abnormality. For example, for certain abnormalities associated with the body part, such as tumors, hemorrhages, etc., in addition to or alternatively to the variations in the intensity of the targeted location (or region) of the simulated lesion, the simulated lesion may push or displace tissues surrounding the simulated region because of the mass effect.

To simulate this mass effect, the computing device 104 may use the contours (e.g., boundaries or outlines) of the lesion mask (e.g., sometimes referred to as abnormality mask) to compute/calculate the gradients around the structure and/or determine/estimate the direction of the deformation. The gradient around the structure and the direction of the deformation can be a part of a deformation field. The mass effect can reflect the effect of the lesion to the surrounding healthy tissues at a certain point in time, for instance, pushing of healthy tissue outwards because of the newly formed lesion mass. For example, the computing device 104 can create the deformation field, for instance, by sampling the displacement vector value for each voxel from a Gaussian distribution. The computing device can smooth out the deformation field by applying a Gaussian kernel spatially. The computing device can adjust or modify the strength/level/magnitude of the deformation, and/or the proportion of the size change to achieve a realistic appearance or effect caused by the abnormality growth. The computing device can constrain the deformation within the simulated mask of the anatomical region, such that the deformation affects the soft tissue, without affecting the hard tissue, for example. In various cases, the computing device 104 can use gradient information of the image to determine the direction of the mass effect. The image gradient direction may be defined as the local difference (e.g., |Img[x,y]−Img[x−1,y]|, |Img[x,y]−Img[x, y−1]|), which may be similar to a local tangent line in 2D. The computing device 104 may utilize other approaches or techniques to determine the gradient, not limited to those described herein. Hence, according to the gradient information, the computing device 104 can determine the deformation direction and/or the strength of the pixels for the simulation. Accordingly, for mass effect simulation, responsive to applying the mass (e.g., simulated lesion) at the selected location, to simulate the mass effect, the computing device 104 can calculate the distortion direction and strength for various local pixels or voxels (e.g., neighboring pixels or voxels) near the lesion mask, and apply such deformation field to the image to simulate the distortion caused by the mass effect.

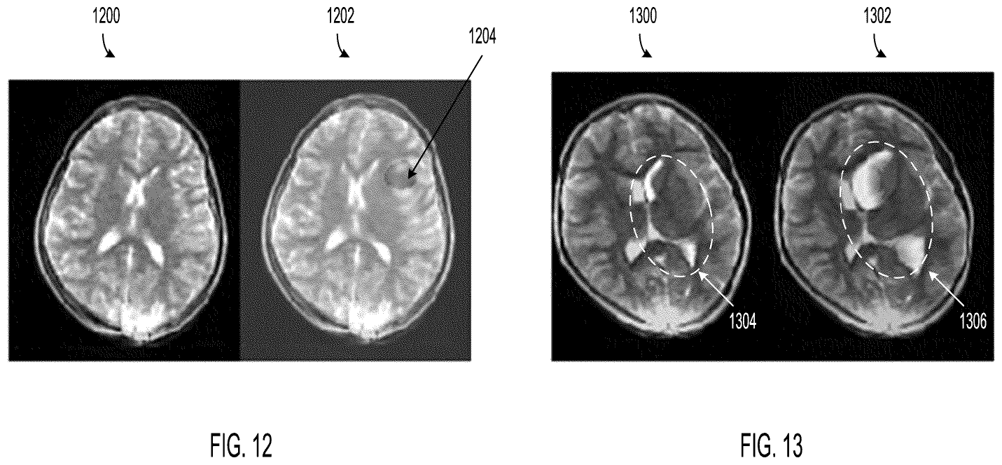

FIG. 12 depicts an example of a simulated mass effect around a mask, in accordance with one or more implementations. As shown, example image 1200 can present the body part without the simulated lesion. Responsive to applying the simulated lesion to the selected location, the computing device 104 may generate example image 1202, which can include the simulated lesion 1204 (e.g., lesion mask). In the example image 1202, the soft tissues surrounding the simulated lesion may be affected by the mass effect caused by the growth of the abnormality.

### Example Data-Driven Configuration

In some configurations, the computing device 104 can utilize the data-driven configuration for simulating the appearance of the simulated lesion. By utilizing the data-driven appearance simulation, the computing device 104 can be configured to inject the simulated lesion into the image (e.g., input image or first image) using at least one ML technique. For example, the computing device 104 can execute a model to receive input data including but not limited to at least one image (e.g., the input image or image patch) to inject the simulated lesion, and a simulated lesion mask (e.g., sometimes referred to as a pathology mask) obtained using at least one of the techniques for simulating the lesion mask, discussed herein. The model can be configured to generate an output image, such as the input image injected with the simulated lesion.

In some implementations, to perform the lesion injection to the input image, the computing device can utilize one or more ML models, such as but not limited to a first model, a second model, and/or a third model. In some cases, these models may be independent models trained or operated using respective ML techniques. In some other cases, these models may be a part of a single model (e.g., cyclicGAN, denoising diffusion probabilistic models (DDPM), etc.), for instance, trained or operated using at least one ML technique, such as at least one of deep learning, neural networks, convolution neural networks, GANs, etc.

For example, the first model can operate as a conditional generator. For example, the first model can take the input image (e.g., labeled as “P”) and the simulated lesion mask (e.g., labeled as “L”). The first model can inject the simulated lesion mask into the input image to generate an output image (e.g., labeled as “Z”). For instance, to perform the injection, a neural network (e.g., labeled as N1, which may be a part of the first model) can be trained to take the input image P as input. The neural network N1 may be a convolutional network. Responsive to obtaining the input image P, the input image P can traverse a series of convolutional layers, for instance, to generate one or more images with the simulated lesion. The process for training the network can be described herein. In some cases, during the training of the neural network N1 (or the first model), other models (e.g., discriminator model) can be trained, such as simultaneously or utilizing a similar training process. In some implementations, during inference (e.g., when the data-driven approach is to be used for injecting the simulated lesions), the first model (e.g., the generator) can be used without at least one other model, such as without the discriminator model.

The second model can operate as a discriminator configured to distinguish between images. For example, the second model can obtain the output image Z from the first model and at least one reference image from a dataset that includes a real (non-simulated) lesion. The second model can perform various operations to distinguish between the output image Z and the real distribution, such as distinguishing between the simulated lesion mask L in the output image Z and the real lesion distributed in the reference image. The distinction may be a part of the training process. For example, the distinction can be performed using a standard GAN framework, such as given a set of healthy images (e.g., patches) {L_H1 . . . L_HN} and a set of pathology images (e.g., patches) {L_A1 . . . L_AN}, the healthy patches can be fed to the neural network N1, which can output {Z1 . . . ZN}. The discriminator (e.g., the second model) can take {Z1 . . . ZN} from the output of the neural network N1 and {L_A1 . . . L_AN} in any order. The discriminator can classify whether the image patch was generated by the generator or whether the image patch was the real sample (e.g., from the L_A's patches). If the discriminator provides an incorrect classification (e.g., compared classification to expected results), backpropagation can be used to (e.g., further) train the discriminator. Otherwise, the computing device 104 can determine that the discriminator has been trained for deployment. In various cases, the first model and the second model can be trained simultaneously.

The third model can operate as a segmenting component configured to segment (or attempt to segment) the simulated lesion mask L from the output image Z. By segmenting the simulated lesion mask L, the computing device 104 can provide the segmented simulated lesion mask L as an annotation for purposes of training or improving the performance of a certain model (e.g., models used for segmentation, which can be trained using the simulated data). The segmentation process may be an extension of the discriminator process (e.g., training for the classification of real images compared to simulated images). The various training discussed herein can be for ensuring that the model inserts a shape specified according to the lesion mask L. In some implementations, to enforce the model to insert the specified shape, a loss function can be added to penalize the model if the inserted lesion shape is different from the lesion mask L, for example. An additional segmenter can be utilized for this training, for instance, by attempting to segment the lesion from the inserted simulated lesion of the output image Z. If the output segmentation of the third model, e.g., labeled as Lz, is different from the lesion mask L, backpropagation can be used to train the generator (e.g., the first model), such that the generator can generate a patch more closely resembling the lesion mask L in a subsequent execution cycle. The loss function can include at least CE (L, segmentor (generator (P))), where CE corresponds to cross-entropy, among others. Additionally or alternatively, DDPM can be trained. Responsive to training the DDPM, the DDPM can be configured to perform feature generation conditioned on input patches (e.g., input image P). The DDPM can be trained using training objectives based on image denoising, among others.

In some implementations, these models (e.g., the first, second, and third models) can be jointly trained, for instance, simultaneously training the models using shared information and/or learning from each other during the training process. In some cases, the third model for segmenting the simulated mask L from the output image Z can be pre-trained using historical datasets for segmenting the lesion from other healthy tissues within the image. In some cases, one or more of these models may be trained on at least one other external computing device. In this case, the computing device 104 can obtain/receive the trained model(s) from the external computing device. In some aspects, the computing device 104 may utilize a fourth model, for instance, to perform a mapping operation from the output image Z to the input image P. For instance, by incorporating the fourth model, the computing device 104 can obtain the ability to perform an image-to-image translation with a certain framework, e.g., cycleGAN framework.

In some implementations, the computing device 104 can replace the third model with at least one or multiple different types of loss functions (e.g., CE (L, segmentor (Z)), etc.), such as to ensure the simulated lesion area can be identified in the generated image. In this case, instead of using the segmentator (e.g., the third model), the computing device 104 may perform another approach, such as computing local correlation to ensure that the simulated lesion portion of the simulated lesion image (e.g., output image Z) has a relatively high correlation with the input lesion mask L. For example, the type of loss functions may include but are not limited to at least one of clustering loss, local cross-correlation, dice loss, focal loss, etc. For instance, the computing device 104 can utilize the clustering loss function to identify values of the pixels or voxels, where the values within the simulated lesion are relatively proximate to the mean (pixel or voxel) value, and relatively distant from the mean value in regions outside the simulated lesion. In another example, by utilizing the local cross-correlation function, the computing device 104 can identify or determine the local correlation between the input image P and the simulated mask L. The local correlation may be in a range from 0 to 1, for example. A relatively high local correlation, such as at least 0.7, can indicate that the simulated mask L has similar or relatable patterns, pixel or voxel values (e.g., contrasting or intensity features), among other features as the input image P (e.g., surrounding tissues). The relatively high local correlation can represent or indicate a relatively high fidelity simulation, which may allow the computing device 104 to use the output image Z for model training and performance improvement purposes. A relatively low local correlation, such as below 0.7, can indicate that the simulated mask L does not appear to have patterns or features relatable to certain tissues from the input image P. The relatively low local correlation can indicate a relatively low fidelity simulation. In such cases, the computing device 104 may not use the output image Z, for example, as part of improving the model performance.

In some configurations, the computing device 104 can be configured to simulate hydrocephalus or other types of abnormalities. FIG. 13 depicts an example of a hydrocephalus simulation, in accordance with one or more implementations. For example, as shown in example image 1300, the computing device 104 can apply/insert/add a simulation of hydrocephalus (e.g., enlarged ventricles) to the image. The input may include ventricles (e.g., unless missing from the subject). To insert the hydrocephalus, the computing device 104 can distort the present ventricle lesions, such as to synthetically enlarge these ventricle lesions. The enlarged ventricle lesions may be representative of or resemble hydrocephalus cases. Responsive to applying the simulated hydrocephalus, the computing device 104 can initiate the mass effect simulation operations, such as described in conjunction with at least FIG. 12. The mass effect simulation can be shown in example image 1302. By executing the mass effect simulation, the computing device 104 can simulate the abnormalities associated with the shape deformation, e.g., of lateral ventricles for hydrocephalus in this case. In further examples, the computing device 104 can compute the deformation based on historical data including historical patterns observed for the corresponding type of abnormality (e.g., in this case, hydrocephalus). The computing device 104 can apply the deformation based on the gradients around the contour of the annotated lateral ventricles, which may be parts of the separated territories of the brain. In various cases, the deformation for each ventricle can be computed/determined and applied independently and/or sequentially for simulating the mass effect. As shown, as a result of the mass effect, the hydrocephalus can be grown from portion 1304 of the example image 1300 to portion 1306 of the example image 1302.

In some cases, the computing device 104 may apply healthy tissue similar to the hydrocephalus simulation. For instance, instead of simulating the hydrocephalus, the computing device 104 can inject healthy tissue at the location, determine the shape of pathological distortion, and simulate mass effect to distort the local voxels.

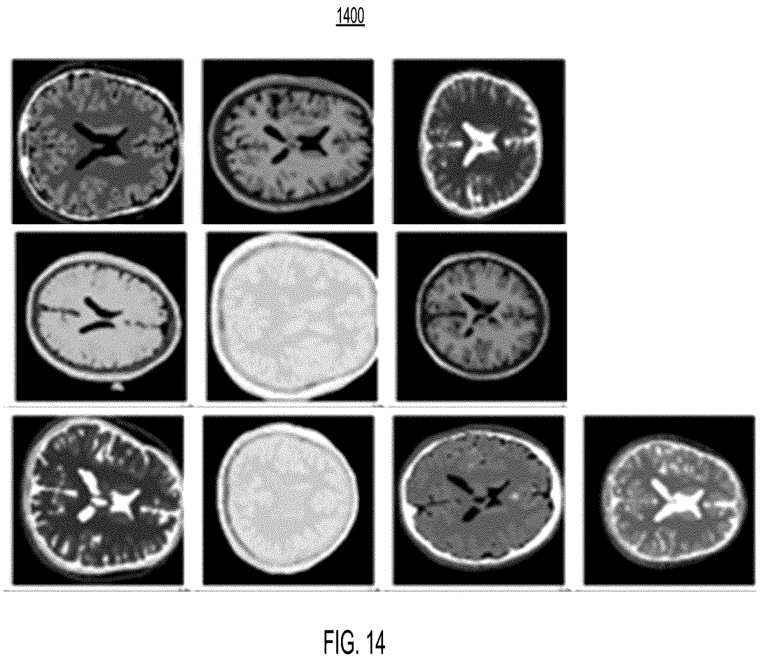

In various configurations, the computing device 104 can simulate the contrast for the image. FIG. 14 depicts an example of MR images 1400 with different contrast, in accordance with one or more implementations. For example, with the segmentation maps of various anatomical regions (or subregions) of the body parts and one or more sequence parameters, the computing device 104 can simulate images of different contrasts. Examples of the sequence parameters can be described herein. As shown in the example MR images 1400 different anatomical regions may be represented in different contrasts. Further, depending on the sequence parameters, individual tissues can be represented in different contrast, thereby simulating variations in the images captured by the one or more imaging devices, for instance, using different configurations/settings.

The potential regions associated with the image can include, but are not limited to, at least one of the background of the image, CSF cerebrospinal fluid, gray matter (GM), white matter (WM), fat, muscle, skull, skin, vessels, dura, marrow, left cerebral white matter, left cerebral cortex, left lateral ventricle, left inferior lateral ventricle, left cerebellum white matter, left cerebellum cortex, left thalamus, left caudate, left putamen, left pallidum, third ventricle, fourth ventricle, brain-stem, left hippocampus, left amygdala, left accumbens area, left ventral diencephalon (DC), right cerebral white matter, right cerebral cortex, right lateral ventricle, right inferior lateral ventricle, right cerebellum white matter, right cerebellum cortex, right thalamus, right caudate, right putamen, right pallidum, right hippocampus, right amygdala, right accumbens area, right ventral DC, etc. In some cases, the subregions can be any pathology subregions. Each brain region can be represented as part of a 2D image or a 3D image of a probability map, where each pixel can correspond to a respective value from 0 to 1 (e.g., normalized). The value 0 can represent the lowest contrast and the value 1 can represent the highest contrast, or vice versa. The size of the image can range from [32, 32, 32] to [512, 512, 512], among other sizes.

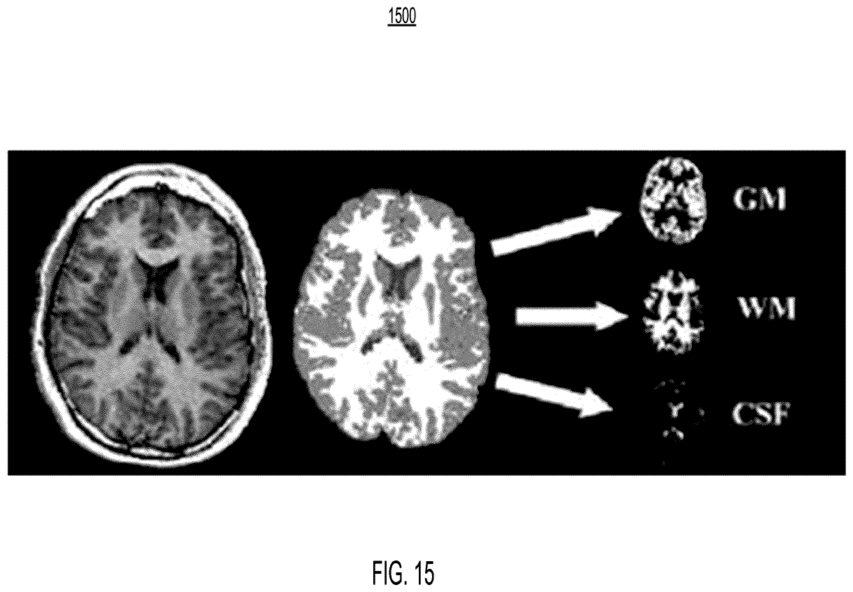

FIG. 15 depicts an example of tissue region probability map 1500 for GM, WM, and CSF, in accordance with one or more implementations. In some aspects, the probability map can indicate the probability of a specific brain region or structure being present at a particular location in the image, such as described in conjunction with at least FIG. 15. In some cases, the probability maps from different regions (e.g., from at least one of GM, WM, or CSF regions) may overlap in pixel locations with each other.

In some implementations, each tissue label can have one or more tissue-specific parameters, such as T1, T2, T2*, and/or PD, among others. The tissue parameter may change depending on the magnetic field strength associated with the image (e.g., input image or simulated image). For example, the tissue-specific parameters for an image associated with a magnetic field strength of 64 mT can be different from the tissue-specific parameters for the same image associated with a magnetic field strength of 1.5 T. Each sequence can have at least one sequence-specific parameter, including but is not limited to relaxation time (TR), echo time (TE), inversion time (TI), flip angle, etc. An example of certain sequence-specific parameters can include, for example FLAIR sequence, TR=3000 ms, TE=80 ms, flip angle=90 deg, and/or TI=1700 s.

The computing device 104 can utilize at least one or a combination of signal formulas/equations to generate or simulate the image contrast. The signal equation can include but is not limited to spin-echo, spoiled gradient echo, rewound gradient, time-reversed gradient echo, and/or random. These example signal equations can be provided in the following examples:

$$Spin - echo : S = PD ( 1 - 2 e \hat{} ( - TI / T 1 ) + e \hat{} ( - TR / T 1 ) ) e \hat{} ( - TE / T 2 )$$

$$Spoiled gradient echo : S = PD * sin ( a ) ( 1 - e \hat{} ( - TR / T 1 ) ) e \hat{} ( - TE / T {2}^{*} )$$

$$1 - cos ( a ) e \hat{} ( - TR / T 1 )$$

$$Rewound gradient : S = PD * sin ( a ) ( 1 - e \hat{} ( - TR / T 1 ) ) e \hat{} ( - TE / T {2}^{*} )$$

$$[ ( 1 - cos ( a ) e \hat{} ( - TR / T 1 ) - e \hat{} ( - TR / T 2 ) ) ( e \hat{} ( - TR / T 1 ) - cos ( a ) ) ]$$

$$Time - reversed gradient echo : S = pde \hat{} ( - 2 ( 2 * TR - TE ) / T 2 )$$

$$Random : S = pd * uniform ( 0 , 1 )$$

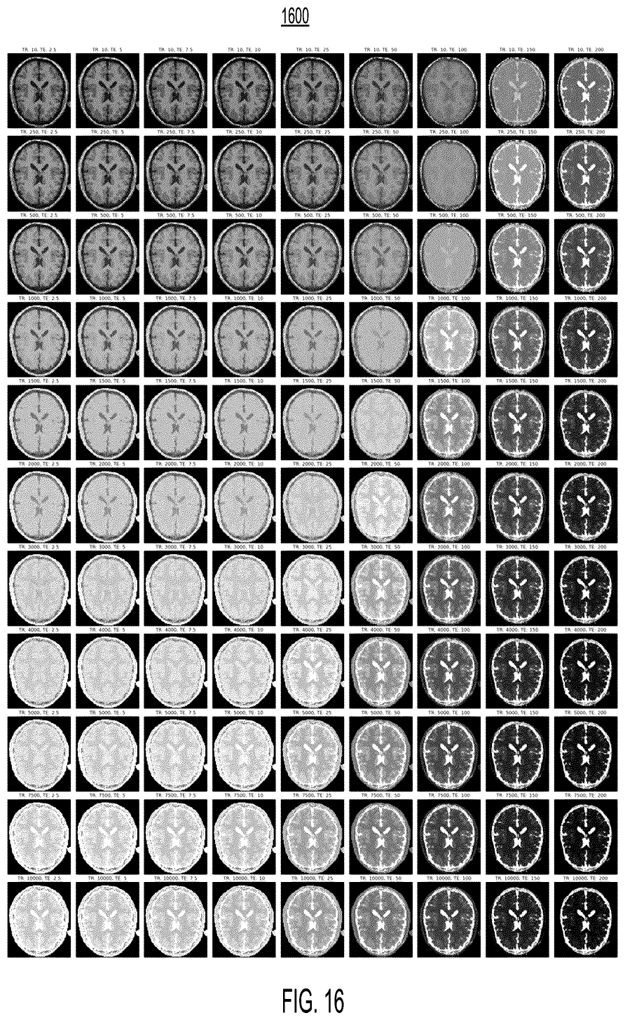

The computing device 104 can generate images with different contrasts as follows. FIG. 16 depicts an example of the same brain using a spin echo signal equation and generating different contrast according to different TR, TE, and/or TI, among other parameters, in accordance with one or more implementations. The example images 1600 of FIG. 16 include the same brain image generated by the computing device 104 with different contrasts. For example, the computing device 104 can select or use the input image, including an image volume, for generating different contrasts. The computing device 104 can select a number of tissue regions (e.g., areas, such as WM, GM, CSF, etc., where one or more of the areas can be selected by the user 102 or randomly selected) to use. In some cases, the tissue regions can include or be similar to anatomy masks. In some cases, contrast simulation can be used to determine the pixel value for each of these regions. The computing device 104 can generate a final image (e.g., output image) by adding these region masks with supplied/provided intensity values based on the contrast simulation. The computing device 104 can select at least one signal equation and one or more sequence parameters, such as TR, TE, TI, and/or flip angle, among others. In some cases, the computing device 104 may select the signal equation randomly. In some other cases, the computing device 104 may sequentially select the signal equation from a list of signal equations. In this case, different signal equations may be selected for each cycle of the image generation process. In yet some other cases, the computing device 104 may receive instructions from the user 102 to utilize at least one predetermined signal equation. For purposes of providing examples, the echo signal equation can be used to generate the example images 1600, although other signal equations can be utilized similarly herein. For each tissue, the computing device 104 can load at least one of the 2D or 3D segmentation probability maps and the tissue parameters. For individual pixels within the probability map, the computing device 104 can compute a signal value (e.g., labeled as “S”) based on the signal equation, tissue parameters, and sequence parameters. Responsive to the computation, the computing device 104 can generate an S-map for each tissue, where the S-map can be the same size as the input segmentation map. With various tissues, the computing device 104 can generate a number of S-maps. Accordingly, the computing device 104 can combine or aggregate the S-maps for the tissues to generate or yield a (e.g., final) combined image. The computing device 104 can reiterate the process to generate other contrasts for the input image, for instance, using different values for the sequence parameters or different sequence parameters.

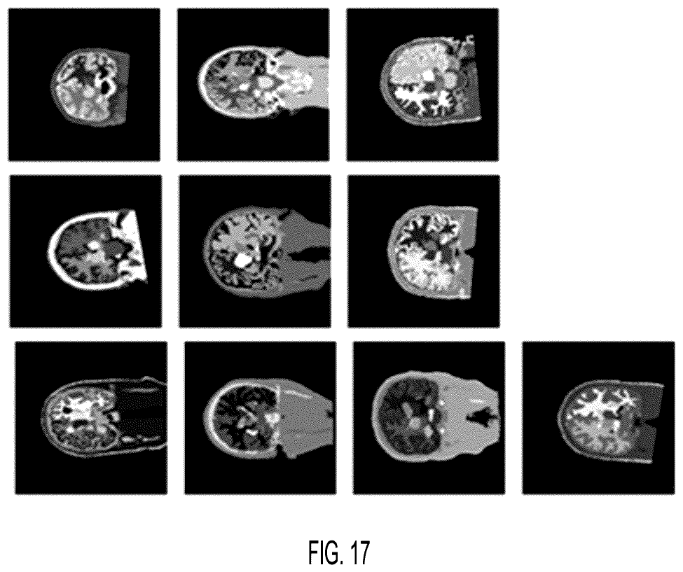

The computing device 104 may use another signal equation to generate images with different contrasts. For example, FIG. 17 depicts an example of contrast generated using the random signal equation, in accordance with one or more implementations. In this case, the computing device 104 can utilize similar operations, such as described in conjunction with at least FIG. 16, to generate example images 1700. For example, instead of the spin echo equation of FIG. 16, the computing device 104 can utilize the random signal equation for generating the example images 1700. The use of the signal equations may not be restricted to any particular brain region, such as shown in the example images 1700 including various regions of the brain. For instance, the computing device 104 can generate different contrasts using the random signal equation for any region. Further, the computing device 104 may use other signal equations (e.g., involving tissue parameters and/or sequence parameters) for image generation, not limited to those described hereinabove.

For purposes of example, the contrast simulation can involve an injection of the simulated lesion. In some other examples, the contrast simulation can involve an injecting of healthy tissue segments into the image of the body part. In such cases, the computing device 104 can overwrite the values of certain pixels or voxels based on at least one signal equation (e.g., sometimes referred to as a contrast equation). In some cases, the computing device 104 may use the simulated contrast for lesion appearance simulation, where the intensity value of the simulated lesion may be determined using the signal equation, for example. In this case, the computing device 104 may use the pixels or voxels of the image (e.g., the first image or the input image) with or without the intensity values of these pixels or voxels. In certain aspects of using the simulated contrast, the computing device 104 may utilize metadata information of the region/segment of the simulated lesion, such as T1 relaxation time (T1) and/or T2 relaxation time (T2) values, for lesion appearance simulation. For instance, each tissue can include a respective tissue property, such as proton density (PD), T1, T2, T2* relaxation, etc. The metadata can include one or more of the tissue properties. These constants can be provided to the signal equations, for instance, to determine the pixel values for various pixels within the tissue regions (e.g., the simulated lesion), or in some cases, as part of the healthy tissue regions.

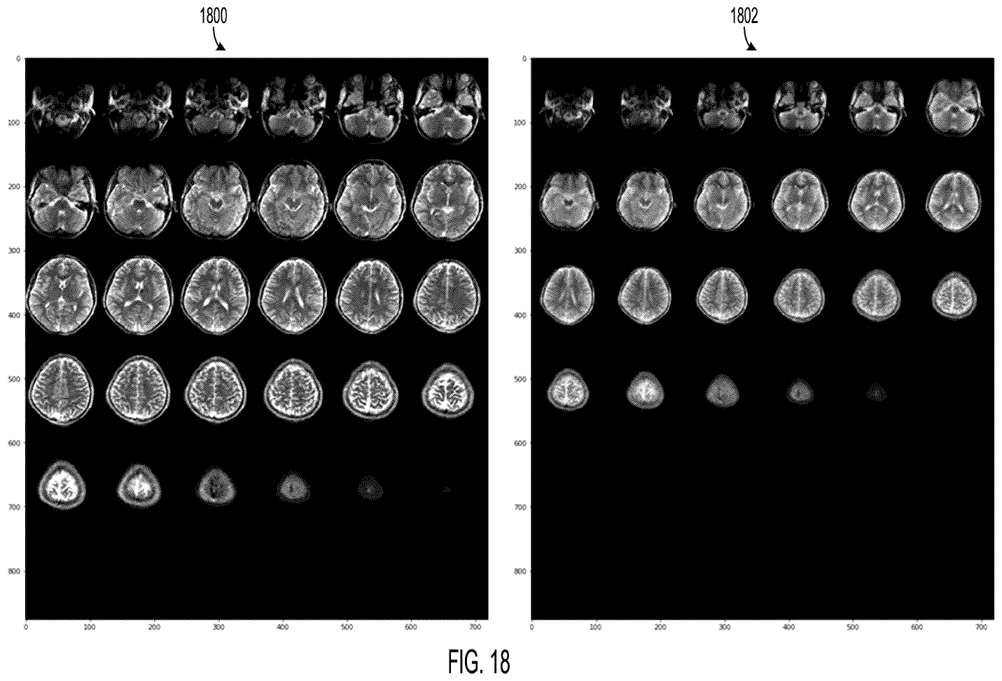

In some configurations, the computing device 104 can perform pediatric simulations to simulate images of the body part of the subject at various development stages, such as from infant to adult. FIG. 18 depicts an example of pediatrics simulations 1800, 1802, in accordance with one or more implementations. For example, to perform the pediatric simulations, the computing device 104 can obtain datasets of a variety of paired adult and neonatal images (e.g., brain MRI scans). These pairs of images can be references for the transformation of adult scans into neonatal scans. The computing device 104 can apply contrast matching (or historical matching) between the neonatal scans and the adult scans, such as matching the neonatal scans to the adult scan or vice versa. Applying the histogram matching can reduce the GM and/or WM contrast in the adult scans (or increase the GM and/or Wm contrast in the neonatal scans) to improve the resemblance of the contrast shown in neonatal scans (or in the adult scans). Subsequently, the computing device 104 can perform body part (e.g., brain) resizing by compressing (or squeezing) the adult scan to match the size of the neonatal scan. Additionally or alternatively, the computing device 104 may resize the brain by stretching the neonatal scan to match the size of the adult scan, for example. By performing the resizing, the computing device 104 can replicate the size and/or shape of the neonatal brain (or the adult brain depending on the starting images). Responsive to performing the contrast matching and brain resizing, the computing device 104 can generate examples of the transformation from the adult scans to the neonatal scans, or vice versa, as shown in the example images of the pediatrics simulations 1800, 1802.

In some cases, the computing device 104 can simulate healthy tissue utilizing operations similar to the pediatric simulation. For instance, the computing device 104 can perform the pediatric simulation by at least one of injecting shapes (e.g., of the healthy tissue), overriding values, and applying global distortions, such as mass effect, contrast simulation, etc., to achieve contrast and size matching or conformity.

As discussed herein, the computing device 104 can perform structure or image simulations for a variety of pathology types, not limited to a specific pathology type. Some examples of the pathologies that can be simulated using the techniques described herein can include but are not limited to stroke (e.g., large vessel occlusion (LVO)), hemorrhage or hematoma (e.g., intracerebral hemorrhage (ICH), intraparenchymal hemorrhage (IPH), subarachnoid hemorrhage (SAH), subdural hematoma (SDH), or epidural hematoma (EDH)), tumor, hyper-intensity lesions (e.g., white matter hyper-intensity or periventricular hyper-intensity), hypo-intensity lesions, trauma (e.g., traumatic brain injury (TBI)), multiple sclerosis (MS), hydrocephalus, enlargement of any specific brain regions, mass effect/edema, or surgery effects (e.g., resection or removal of the skull or any other anatomy). In some cases, the computing device 104 can utilize the techniques discussed herein to simulate characteristics of Alzheimer's (e.g., cerebral atrophy, such as in the hippocampus and/or temporal lobes, brain volume loss, etc.), Parkinson's (e.g., atrophy of the substantia nigra and/or other brainstem structures), brain abscess (e.g., round lesions with a hyper-intense border and/or hypo-intense center), or meningitis (e.g., thin layer of hyper-intensity along the surface of the brain), among others. In various cases, certain pathologies (e.g., tumors) may have relatively complex appearances, such as necrosis, tumor core, edema, etc.

In some configurations, one or more of the techniques described herein, for instance, lesion mask simulation and/or lesion appearance simulation may be applied multiple times/iterations such that a number of lesions are injected on top of one another. By applying multiple simulated lesions or appearances, the computing device 104 can create images with simulated lesions having relatively more complex and diverse appearances. Accordingly, the computing device 104 can provide the simulated structures or images as training data to train (or further train) one or more models for separating healthy structures from abnormal structures, thereby improving the performance of the model to diagnose and treat a variety of health conditions. The computing device 104 can provide the simulation data to other processing devices for improving model performance or use the simulation data to train at least one local model.

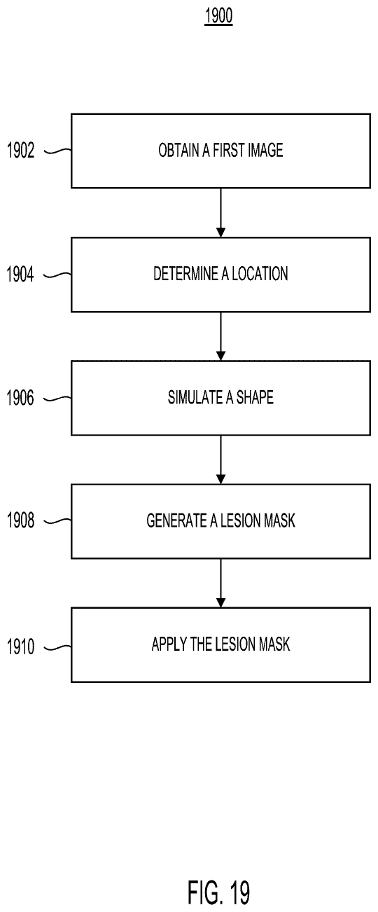

Referring to FIG. 19, depicted is a flowchart of an example method 1900 for simulating structures or images, in accordance with one or more implementations. The method 1900 may be executed using any suitable computing system (e.g., the computing device 104, the controller 106 of FIG. 1, the computing system 2000 of FIG. 20, etc.). It may be appreciated that certain steps of the method 1900 may be executed in parallel (e.g., concurrently) or sequentially, while still achieving useful results. The method 1900 may be executed to simulate one or more structures or images, as described herein.

At operation 1902, the method 1900 can include obtaining, such as by a computing device (e.g., computing device 104) or a controller (e.g., controller 106), a first image of a subject. The first image may be a first MR image or other types of images captured by an imaging device, such as a CT image, ultrasound image, etc.

At operation 1904, the method 1900 can include determining, by the computing device, a location for simulating a structure within the first image. The structure may include or correspond to healthy tissue or a lesion. For purposes of examples herein, the structure to be simulated or for simulating an image can include a lesion. In some other aspects, the structure to be simulated can be healthy tissue. In some implementations, to determine the location, the computing device can identify at least one anatomical region associated with a body part of the subject. For purposes of example, the anatomical region associated with the first image can be at least a portion of the brain of the subject. The computing device can extract (or separate), using or from the identified at least one anatomical region, various territories associated with the first image. Extracting the territories may refer to classifying the territories from the first image, such as described in conjunction with at least the operation 210 of FIG. 2, for example. Based on the extracted territories, the computing device can select at least one first territory associated with the first image as the location for a mask (e.g., a lesion mask, structure mask, abnormality mask, or tissue mask). This mask may be a first mask simulated by the computing device. As discussed herein, the computing device 104 can apply the mask to the first image, at least in part, to generate another image (e.g., a second image), where the second image includes at least a portion of the first image with the simulated mask.

In some implementations, subsequent to selecting the at least one first territory as the location, the computing device can select at least one second territory associated with the first image as a second location for another mask. In such cases, the computing device can apply another mask (e.g., a second mask) to the first image to generate a third image, for example. The third image can include at least a portion of the first image with the simulated masks (e.g., masks in the first territory and the second territory). In some cases, the second mask can be a part of the first mask (e.g., extension of the first mask).

In some implementations, the computing device may receive an indication of at least one territory associated with the first image, such as from the user (e.g., the user 102). In this case, the computing device 104 can use the at least one received territory as the location to simulate the lesion.

At operation 1906, the method 1900 can include simulating, by the computing device, according to the location, a shape for the structure. For example, the computing device can simulate the shape of the structure based on or according to the location (e.g., at least one territory) associated with the first image. The shape and/or size of the structure can be based on the associated territory or location of the body part.

In some implementations, to simulate the shape, the computing device can generate an elliptical shape according to one or more parameters. The one or more parameters may include a long axis or a short axis of the first image defining a dimension of the structure, for example. In some cases, the one or more parameters can be based on the location at which the shape is to be simulated. The computing device may apply an elastic distortion to the elliptical shape to simulate the shape of the structure. The shape simulation may be described in conjunction with, but not limited to FIG. 6.

At operation 1908, the method 1900 can include generating, by the computing device, a mask (e.g., the first mask) according to the location and the shape for the structure. In some implementations, the computing device 104 can generate the mask having the shape and/or size, such as described in conjunction with, but not limited to FIG. 6. The mask can be a 2D mask or a 3D mask including multiple 2D masks.

In some implementations, to generate the mask, the computing device can place or provide a seed to the first image at the location, such as described in conjunction with at least the region-growing technique/approach. The seed can represent a starting point for growth of the mask. The computing device can grow at least one region around the seed using at least one region-growing algorithm. The region-growing algorithm can include but is not limited to at least one of: region growing, region merging, split and merge, watershed transform, connected component labeling, or graph-cut segmentation. The computing device can iteratively grow the region around the seed (e.g., to neighboring pixels or voxels) until at least one condition is satisfied, such as the maximum dimension of the mask, the number of iterations, etc. Based on the grown seed (or regions around the seed), the computing device can generate the mask for lesion simulation.

In some implementations, the seed can be provided at at least one voxel having a first intensity (e.g., voxel intensity). The computing device can grow the region around the seed to at least one neighboring voxel having a second intensity, wherein the second intensity is substantially similar to the first intensity. The computing device may prevent region growth to other regions with an intensity substantially different from the first intensity, for example, to maintain the lesion in the same anatomical region. In some cases, multiple seeds can be provided to multiple voxels as starting points for the growth of at least one mask.

In some implementations, to generate the mask, the computing device can obtain historical masks associated with historical images from one or more subjects, such as described in conjunction with at least the data-driven pathology mask generation procedures. The historical masks may refer to real lesions captured by at least one imaging device. In this case, the computing device can train a model using at least one ML technique based on the historical masks. The model can learn the patterns, features, characteristics, or other details associated with the historical masks. Using the model, the computing device can generate the mask for the location according to the historical masks. The computing device can refine, modify, or perform any other updates the mask based on a comparison between the generated mask and the historical masks associated with the location for simulating the structure. For instance, based on the similarities or discrepancies in the features between the mask and the historical mask(s), the computing device can provide one or more corrections to the generated mask to increase the fidelity of the simulation.

In some implementations, to generate the mask, the computing device may determine the appearance of the mask (e.g., of the simulated lesion). The appearance of the mask may include but is not limited to the intensity of the one or more voxels of the first image. The intensity can refer to the pixel or voxel intensity. The appearance may include other features discussed herein, such as contrasts, noises, patterns, etc.

In some implementations, to determine the appearance, the computing device can select an aggregated (e.g., average, mean, median, etc.) pixel intensity of the mask. The computing device can apply at least one pattern to the mask. The at least one pattern can include but is not limited to at least one of: edema pattern, hemorrhagic pattern, necrotic pattern, cystic pattern, inflammatory pattern, tumoral pattern, and/or ischemic pattern. Additionally or alternatively to applying the at least one pattern, the computing device can simulate at least one noise for the mask. The at least one noise can include but is not limited to acquisition noise, Gaussian noise, etc. The aggregated pixel intensity, at least one patter, and/or at least one noise can be part of the appearance of the mask. The computing device can perform other operations, such as described in conjunction with the lesion appearance simulation of the operation 224 of at least FIG. 2.

In some implementations, to generate the mask, the computing device can simulate a shape deformation associated with the shape, such as described in conjunction with but not limited to the hydrocephalus simulation of least FIG. 13. In this case, simulating the shape deformation may correspond to simulating hydrocephalus for the body part of the subject. The computing device can generate the mask according to the location and the shape deformation for the structure.

At operation 1910, the method 1900 can include applying, by the computing device, the mask to the first image (e.g., input image) to generate or simulate a second image (e.g., output image) simulating the structure. The second image may be a second MR image or other types of image similar to the first image.

In some implementations, to determine the appearance and apply the mask, the computing device can perform features or operations, for instance, described in conjunction with at least a part of the lesion appearance simulation. For example, the computing device can provide the generated mask and at least a portion of the first image for applying the mask as inputs to a model trained using at least one machine learning technique. Using the model, the computing device can generate a third image comprising at least the portion of the first image and the generated mask (e.g., injected into the first image). The computing device can compare, using the model, the third image to at least one historical image. The at least one historical image can have a second mask (e.g., corresponding to or including historical structure or real lesion) at the location with a second shape similar to the shape of the mask. In some cases, the second mask may have different shape compared to the mask. Based on the comparison, the computing device can identify features or details of the structure in the third image comparable to a historical structure (e.g., real lesion) of the historical image and/or features not representative of the historical structure. According to the comparison, the computing device can update the third image to enhance the fidelity of the simulated structure and/or image (e.g., third image in this case).

In some implementations, to apply the mask, the computing device can simulate a mass effect around the mask, such as described in conjunction with but not limited to at least one of FIGS. 12-13. In this case, the computing device can apply the mask to the first image with the simulated mass effect to generate the second image simulating the structure. In some cases, the mass effect may be a part of the appearance of the mask.

In some implementations, to apply the mask, the computing device can identify one or more anatomical regions associated with a body part of the subject. Each of the anatomical regions can be represented as a 2D image or a 3D image. The computing device can simulate contrast for the first image based on at least one of the anatomical regions and one or more sequence parameters. The computing device can utilize at least one signal equation (e.g., sometimes referred to as a contrast equation) for simulating the contrast, including but not limited to at least one of: spin-echo, spoiled gradient echo, rewound gradient, time-reversed gradient echo, and/or random equation. The one or more sequence parameters can include at least one of relaxation time, echo time, inversion time, and/or flip angle, among others. Different contrast details for images can be generated with different sequence parameters. The computing device can apply the mask and the simulated contrast to the first image to generate the second image simulating the structure. In some cases, the contrast can indicate at least one of GM, WM, and/or CSF, among others associated with the body part of the subject. In various cases, the contrast can be a part of the appearance of the mask. In such cases, generating the mask can include the computing device determining an appearance of the mask based on the simulated contrast. Examples for simulating the contrast can be described in conjunction with at least one of but not limited to FIGS. 14-17.

In some implementations, the computing device can perform pediatric simulations, such as simulating pediatric images or transforming between adult scans and neonatal scans. The operations for pediatric simulations can be described in conjunction with at least FIG. 18. For example, the computing device can obtain images of a plurality of subjects, comprising at least a fourth image and a fifth image associated with a body part of at least one subject (e.g., adult and/or neonatal subject). The fourth image can be a neonatal image (e.g., neonatal scan) of the body part of the at least one subject, such as from the neonatal subject. The fifth image can be a developed image of the body part of the at least one subject (e.g. adult scan).

For purposes of example, the computing device can be configured to transform adult scan of the fifth image into a simulated neonatal scan, for instance, to simulate a sixth image. In this case, the sixth image can be another neonatal image simulated from the developed image of the body part of the at least one subject. For example, the computing device can conform the fifth image to the fourth image. Conforming the fifth image to the fourth image can include at least one of conforming a first contrast of the fifth image to a second contrast of the fourth image (e.g., contrast matching) and/or conforming a first size of the fifth image to a second size of the fourth image (e.g., size matching). Conforming the first contrast to the second contrast and conforming the first size to the second size can include at least one of: injecting one or more shapes to the fifth image according to the fourth image; overriding one or more values associated with pixels or voxels of the fifth image according to the fourth image; and/or applying one or more distortions to the fifth image according to the fourth image. By conforming one or more features of the fifth image to the fourth image, the computing device can transform the fifth image to the sixth image (or simulate the sixth image) representing the neonatal scan simulated from the adult scan, for example.

FIG. 20 illustrates a component diagram of an example computing system suitable for use in the various implementations described herein, according to an example implementation. In a non-limiting example, the computing system 2000 may implement a computing device 104 or controller 106 of FIG. 1, or various other example systems and devices described in the present disclosure.

The computing system 2000 includes a bus 2002 or other communication component for communicating information and a processor 2004 coupled to the bus 2002 for processing information. The computing system 2000 also includes main memory 2006, such as a RAM or other dynamic storage device, coupled to the bus 2002 for storing information, and instructions to be executed by the processor 2004. Main memory 2006 may also be used for storing position information, temporary variables, or other intermediate information during execution of instructions by the processor 2004. The computing system 2000 may further include a ROM 2008 or other static storage device coupled to the bus 2002 for storing static information and instructions for the processor 2004. A storage device 2010, such as a solid-state device, magnetic disk, or optical disk, is coupled to the bus 2002 for persistently storing information and instructions.

The computing system 2000 may be coupled via the bus 2002 to a display 2014, such as a liquid crystal display, or active matrix display, for displaying information to a user. An input device 2012, such as a keyboard including alphanumeric and other keys, may be coupled to the bus 2002 for communicating information, and command selections to the processor 2004. In another implementation, the input device 2012 has a touch screen display. The input device 2012 may include any type of biometric sensor, or a cursor control, such as a mouse, a trackball, or cursor direction keys, for communicating direction information and command selections to the processor 2004 and for controlling cursor movement on the display 2014.

In some implementations, the computing system 2000 may include a communications adapter 2016, such as a networking adapter. Communications adapter 2016 may be coupled to bus 2002 and may be configured to enable communications with a computing or communications network or other computing systems. In various illustrative implementations, any type of networking configuration may be achieved using communications adapter 2016, such as wired (e.g., via Ethernet), wireless (e.g., via Wi-Fi, Bluetooth), satellite (e.g., via GPS) pre-configured, ad-hoc, LAN, WAN, and the like.

According to various implementations, the processes of the illustrative implementations that are described herein may be achieved by the computing system 2000 in response to the processor 2004 executing an implementation of instructions contained in main memory 2006. Such instructions may be read into main memory 2006 from another computer-readable medium, such as the storage device 2010. Execution of the implementation of instructions contained in main memory 2006 causes the computing system 2000 to perform the illustrative processes described herein. One or more processors in a multi-processing implementation may also be employed to execute the instructions contained in main memory 2006. In alternative implementations, hard-wired circuitry may be used in place of or in combination with software instructions to implement illustrative implementations. Thus, implementations are not limited to any specific combination of hardware circuitry and software.

Potential embodiments include, without limitation:

Embodiment AA: A method for simulating structures in images, comprising: obtaining a first image of a subject; determining a location for simulating a structure within the first image; simulating, according to the location, a shape for the structure; generating a mask according to the location and the shape for the structure; and applying the mask to the first image to generate a second image simulating the structure.

Embodiment AB: The method of Embodiment AA, wherein determining the location within the image comprises: identifying at least one anatomical region associated with a body part of the subject; extracting, using the identified at least one anatomical region, a plurality of territories associated with the first image; and selecting at least one first territory associated with the first image as the location for the mask.

Embodiment AC: The method of Embodiment AB, further comprising, subsequent to selecting the at least one first territory, selecting at least one second territory associated with the first image as a second location for another mask for generating a third image.

Embodiment AD: The method of any of Embodiments AA to AC, wherein determining the location and simulating the shape comprises: receiving an indication of at least one territory associated with the first image as the location; and simulating the shape of the structure based on the at least one territory associated with the first image.

Embodiment AE: The method of any of Embodiments AA to AD, wherein generating the mask comprises: providing a seed to the first image at the location; growing at least one region around the seed using at least one region-growing algorithm; and generating the mask based on the grown seed.

Embodiment AF: The method of Embodiments AE, wherein the at least one region-growing algorithm comprises at least one of: region growing, region merging, split and merge, watershed transform, connected component labeling, or graph-cut segmentation.

Embodiment AG: The method of Embodiments AE, wherein the seed is provided at at least one voxel having a first intensity, and wherein the region around the seed is grown to at least one neighboring voxel having a second intensity, wherein the second intensity is substantially similar to the first intensity.

Embodiment AH: The method of any of Embodiments AA to AG, wherein simulating the shape comprises: generating an elliptical shape according to one or more parameters, the one or more parameters comprising a long axis or a short axis of the first image defining a dimension of the structure; and applying an elastic distortion to the elliptical shape to simulate the shape of the structure.

Embodiment AI: The method of any of Embodiments AA to AH, wherein generating the mask comprises: obtaining a plurality of historical masks associated with a plurality of images from one or more subjects; training a model using at least one machine learning technique based on the plurality of historical masks; generating, using the trained model, the mask for the location according to the plurality of historical masks; and refining the mask based on a comparison between the generated mask and the plurality of historical masks associated with the location for simulating the structure.

Embodiment AJ: The method of any of Embodiments AA to AI, wherein generating the mask comprises determining an appearance of the mask associated with at least an intensity of one or more voxels of the first image.

Embodiment AK: The method of Embodiment AJ, wherein determining the appearance comprises: selecting an aggregated pixel intensity of the mask; applying at least one pattern for the mask; and simulating at least one noise for the mask.

Embodiment AL The method of Embodiment AK, wherein the at least one pattern comprises at least one of: edema pattern, hemorrhagic pattern, necrotic pattern, cystic pattern, inflammatory pattern, tumoral pattern, or ischemic pattern.

Embodiment AM: The method of Embodiment AJ, wherein determining the appearance and applying the mask comprises: providing the generated mask and at least a portion of the first image for applying the mask as inputs to a model trained using a machine learning technique; generating, using the model, a third image comprising at least the portion of the first image and the generated mask; and updating the third image based on a comparison of the third image to at least one historical image, the at least one historical image having a second mask at the location with a second shape similar to the shape of the mask.

Embodiment AN: The method of any of Embodiments AA to AM, wherein applying the mask comprises: simulating a mass effect around the mask; and applying the mask to the first image with the simulated mass effect to generate the second image simulating the structure.

Embodiment AO: The method of any of Embodiments AA to AN, wherein the first image is a first magnetic resonance (MR) image and the second image is a second MR image.

Embodiment AP: The method of any of Embodiments AA to AO, wherein generating the mask comprises: simulating a shape deformation associated with the shape; and generating the mask according to the location and the shape deformation for the structure.

Embodiment AQ: The method of Embodiment AP, wherein simulating the shape deformation corresponds to simulating hydrocephalus for the body part of the subject.

Embodiment AR: The method of any of Embodiments AA to AQ, wherein applying the mask further comprises: identifying a plurality of anatomical regions associated with a body part of the subject; simulating contrast for the first image based on at least one of the plurality of anatomical regions and one or more sequence parameters; and applying the mask and the simulated contrast to the first image to generate the second image simulating the structure.

Embodiment AS: The method of Embodiment AR, wherein the one or more sequence parameters comprise at least one of relaxation time, echo time, inversion time, or flip angle.

Embodiment AT: The method of Embodiment AR, wherein each of the plurality of anatomical regions is represented as a 2-dimensional (2D) image or a 3-dimensional (3D) image.

Embodiment AU: The method of Embodiment AR, wherein the contrast indicates at least one of gray matter (GM), white matter (WM), or cerebrospinal fluid (CSF) associated with the body part of the subject.

Embodiment AV: The method of Embodiment AR, wherein simulating the contrast comprises using at least one signal equation, comprising at least one of: spin-echo, spoiled gradient echo, rewound gradient, time-reversed gradient echo, or random.

Embodiment AW: The method of Embodiment AR, wherein generating the mask comprises determining an appearance of the mask based on the simulated contrast.

Embodiment AX: The method of any of Embodiments AA to AW, comprising: obtaining a plurality of images of a plurality of subjects, comprising at least a fourth image and a fifth image associated with a body part of at least one subject; conforming the fifth image to the fourth image; and simulating a sixth image according to the conformed fifth image.

Embodiment AY: The method of Embodiment AX, wherein conforming the fifth image to the fourth image comprises at least one of: conforming a first contrast of the fifth image to a second contrast of the fourth image; and conforming a first size of the fifth image to a second size of the fourth image.

Embodiment AZ: The method of Embodiment AY, wherein the fourth image is a neonatal image of the body part of the at least one subject, the fifth image is a developed image of the body part of the at least one subject, and the sixth image is another neonatal image simulated from the developed image of the body part of the at least one subject.

Embodiment AAa: The method of Embodiment AY, wherein conforming the first contrast to the second contrast and conforming the first size to the second size comprises at least one of: injecting one or more shapes to the fifth image according to the fourth image; overriding one or more values associated with pixels or voxels of the fifth image according to the fourth image; or applying one or more distortions to the fifth image according to the fourth image.

Embodiment BA: A system for simulating structures in images, comprising one or more processors configured to: obtain a first image of a subject; determine a location for simulating a structure within the first image; simulate, according to the location, a shape for the structure; generate a mask according to the location and the shape for the structure; and apply the mask to the first image to generate a second image simulating the structure.

Embodiment CA: A non-transitory computer-readable storage medium storing processor-executable instructions that, when executed by at least one processor, cause the at least one processor to: obtain a first image of a subject; determine a location for simulating a structure within the first image; simulate, according to the location, a shape for the structure; generate a mask according to the location and the shape for the structure; and apply the mask to the first image to generate a second image simulating the structure.

The implementations described herein have been described with reference to drawings. The drawings illustrate certain details of specific implementations that implement the systems, methods, and programs described herein. Describing the implementations with drawings should not be construed as imposing on the disclosure any limitations that may be present in the drawings.

It should be understood that no claim element herein is to be construed under the provisions of 35 U.S.C. § 112(f), unless the element is expressly recited using the phrase “means for.”

As used herein, the term “circuit” may include hardware structured to execute the functions described herein. In some implementations, each respective “circuit” may include machine-readable media for configuring the hardware to execute the functions described herein. The circuit may be embodied as one or more circuitry components including, but not limited to, processing circuitry, network interfaces, peripheral devices, input devices, output devices, sensors, etc. In some implementations, a circuit may take the form of one or more analog circuits, electronic circuits (e.g., integrated circuits (IC), discrete circuits, system on a chip (SOC) circuits), telecommunication circuits, hybrid circuits, and any other type of “circuit.” In this regard, the “circuit” may include any type of component for accomplishing or facilitating achievement of the operations described herein. In a non-limiting example, a circuit as described herein may include one or more transistors, logic gates (e.g., NAND, AND, NOR, OR, XOR, NOT, XNOR), resistors, multiplexers, registers, capacitors, inductors, diodes, wiring, and so on.

The “circuit” may also include one or more processors communicatively coupled to one or more memory or memory devices. In this regard, the one or more processors may execute instructions stored in the memory or may execute instructions otherwise accessible to the one or more processors. In some implementations, the one or more processors may be embodied in various ways. The one or more processors may be constructed in a manner sufficient to perform at least the operations described herein. In some implementations, the one or more processors may be shared by multiple circuits (e.g., circuit A and circuit B may comprise or otherwise share the same processor, which, in some example implementations, may execute instructions stored, or otherwise accessed, via different areas of memory). Alternatively or additionally, the one or more processors may be structured to perform or otherwise execute certain operations independent of one or more co-processors.

In other example implementations, two or more processors may be coupled via a bus to enable independent, parallel, pipelined, or multi-threaded instruction execution. Each processor may be implemented as one or more general-purpose processors, ASICs, FPGAs, GPUs, TPUs, digital signal processors (DSPs), or other suitable electronic data processing components structured to execute instructions provided by memory. The one or more processors may take the form of a single core processor, multi-core processor (e.g., a dual core processor, triple core processor, or quad core processor), microprocessor, etc. In some implementations, the one or more processors may be external to the apparatus, in a non-limiting example, the one or more processors may be a remote processor (e.g., a cloud-based processor). Alternatively or additionally, the one or more processors may be internal or local to the apparatus. In this regard, a given circuit or components thereof may be disposed locally (e.g., as part of a local server, a local computing system) or remotely (e.g., as part of a remote server such as a cloud based server). To that end, a “circuit” as described herein may include components that are distributed across one or more locations.

An exemplary system for implementing the overall system or portions of the implementations might include a general purpose computing devices in the form of computers, including a processing unit, a system memory, and a system bus that couples various system components including the system memory to the processing unit. Each memory device may include non-transient volatile storage media, non-volatile storage media, non-transitory storage media (e.g., one or more volatile or non-volatile memories), etc. In some implementations, the non-volatile media may take the form of ROM, flash memory (e.g., flash memory such as NAND, 3D NAND, NOR, 3D NOR), EEPROM, MRAM, magnetic storage, hard discs, optical discs, etc. In other implementations, the volatile storage media may take the form of RAM, TRAM, ZRAM, etc. Combinations of the above are also included within the scope of machine-readable media. In this regard, machine-executable instructions comprise, in a non-limiting example, instructions and data, which cause a general-purpose computer, special purpose computer, or special purpose processing machines to perform a certain function or group of functions. Each respective memory device may be operable to maintain or otherwise store information relating to the operations performed by one or more associated circuits, including processor instructions and related data (e.g., database components, object code components, script components), in accordance with the example implementations described herein.

It should also be noted that the term “input devices,” as described herein, may include any type of input device including, but not limited to, a keyboard, a keypad, a mouse, joystick, or other input devices performing a similar function. Comparatively, the term “output device,” as described herein, may include any type of output device including, but not limited to, a computer monitor, printer, facsimile machine, or other output devices performing a similar function.

It should be noted that although the diagrams herein may show a specific order and composition of method steps, it is understood that the order of these steps may differ from what is depicted. In a non-limiting example, two or more steps may be performed concurrently or with partial concurrence. Also, some method steps that are performed as discrete steps may be combined, steps being performed as a combined step may be separated into discrete steps, the sequence of certain processes may be reversed or otherwise varied, and the nature or number of discrete processes may be altered or varied. The order or sequence of any element or apparatus may be varied or substituted according to alternative implementations. Accordingly, all such modifications are intended to be included within the scope of the present disclosure as defined in the appended claims. Such variations will depend on the machine-readable media and hardware systems chosen and on designer choice. It is understood that all such variations are within the scope of the disclosure. Likewise, software and web implementations of the present disclosure could be accomplished with standard programming techniques with rule-based logic and other logic to accomplish the various database searching steps, correlation steps, comparison steps, and decision steps.

While this specification contains many specific implementation details, these should not be construed as limitations on the scope of any inventions or of what may be claimed, but rather as descriptions of features specific to particular implementations of the systems and methods described herein. Certain features that are described in this specification in the context of separate implementations may also be implemented in combination in a single implementation. Conversely, various features that are described in the context of a single implementation may also be implemented in multiple implementations separately or in any suitable subcombination. Moreover, although features may be described above as acting in certain combinations and even initially claimed as such, one or more features from a claimed combination may in some cases be excised from the combination, and the claimed combination may be directed to a subcombination or variation of a subcombination.

In certain circumstances, multitasking and parallel processing may be advantageous. Moreover, the separation of various system components in the implementations described above should not be understood as requiring such separation in all implementations, and it should be understood that the described program components and systems may generally be integrated together in a single software product or packaged into multiple software products.

Having now described some illustrative implementations and implementations, it is apparent that the foregoing is illustrative and not limiting, having been presented by way of example. In particular, although many of the examples presented herein involve specific combinations of method acts or system elements, those acts and those elements may be combined in other ways to accomplish the same objectives. Acts, elements, and features discussed only in connection with one implementation are not intended to be excluded from a similar role in other implementations.

The phraseology and terminology used herein is for the purpose of description and should not be regarded as limiting. The use of “including,” “comprising,” “having,” “containing,” “involving,” “characterized by,” “characterized in that,” and variations thereof herein, is meant to encompass the items listed thereafter, equivalents thereof, and additional items, as well as alternate implementations consisting of the items listed thereafter exclusively. In one implementation, the systems and methods described herein consist of one, each combination of more than one, or all of the described elements, acts, or components.

Any references to implementations or elements or acts of the systems and methods herein referred to in the singular may also embrace implementations including a plurality of these elements, and any references in plural to any implementation or element or act herein may also embrace implementations including only a single element. References in the singular or plural form are not intended to limit the presently disclosed systems or methods, their components, acts, or elements to single or plural configurations. References to any act or element being based on any information, act, or element may include implementations where the act or element is based at least in part on any information, act, or element.

Any implementation disclosed herein may be combined with any other implementation, and references to “an implementation,” “some implementations,” “an alternate implementation,” “various implementation,” “one implementation,” or the like are not necessarily mutually exclusive and are intended to indicate that a particular feature, structure, or characteristic described in connection with the implementation may be included in at least one implementation. Such terms as used herein are not necessarily all referring to the same implementation. Any implementation may be combined with any other implementation, inclusively or exclusively, in any manner consistent with the aspects and implementations disclosed herein.

References to “or” may be construed as inclusive so that any terms described using “or” may indicate any of a single, more than one, and all of the described terms.

Where technical features in the drawings, detailed description or any claim are followed by reference signs, the reference signs have been included for the sole purpose of increasing the intelligibility of the drawings, detailed description, and claims. Accordingly, neither the reference signs nor their absence have any limiting effect on the scope of any claim elements.

The foregoing description of implementations has been presented for purposes of illustration and description. It is not intended to be exhaustive or to limit the disclosure to the precise form disclosed, and modifications and variations are possible in light of the above teachings or may be acquired from this disclosure. The implementations were chosen and described in order to explain the principals of the disclosure and its practical application to enable one skilled in the art to utilize the various implementations and with various modifications as are suited to the particular use contemplated. Other substitutions, modifications, changes, and omissions may be made in the design, operating conditions and implementation of the implementations without departing from the scope of the present disclosure as expressed in the appended claims.

What is claimed is:

1. A method for simulating structures in images, comprising:
obtaining a first image of a subject;
determining a location for simulating a structure within the first image;
simulating, according to the location, a shape for the structure;
generating a mask according to the location and the shape for the structure; and
applying the mask to the first image to generate a second image simulating the structure.

2. The method of claim 1, wherein determining the location within the image comprises:
identifying at least one anatomical region associated with a body part of the subject;
extracting, using the identified at least one anatomical region, a plurality of territories associated with the first image; and
selecting at least one first territory associated with the first image as the location for the mask.

3. The method of claim 2, further comprising, subsequent to selecting the at least one first territory,
selecting at least one second territory associated with the first image as a second location for another mask for generating a third image.

4. The method of claim 1, wherein determining the location and simulating the shape comprises:
receiving an indication of at least one territory associated with the first image as the location; and
simulating the shape of the structure based on the at least one territory associated with the first image.

5. The method of claim 1, wherein generating the mask comprises:
providing a seed to the first image at the location;
growing at least one region around the seed using at least one region-growing algorithm; and
generating the mask based on the grown seed.

6. The method of claim 5, wherein the at least one region-growing algorithm comprises at least one of: region growing, region merging, split and merge, watershed transform, connected component labeling, or graph-cut segmentation.

7. The method of claim 5, wherein the seed is provided at at least one voxel having a first intensity, and wherein the region around the seed is grown to at least one neighboring voxel having a second intensity, wherein the second intensity is substantially similar to the first intensity.

8. The method of claim 1, wherein simulating the shape comprises:
generating an elliptical shape according to one or more parameters, the one or more parameters comprising a long axis or a short axis of the first image defining a dimension of the structure; and
applying an elastic distortion to the elliptical shape to simulate the shape of the structure.

9. The method of claim 1, wherein generating the mask comprises:
obtaining a plurality of historical masks associated with a plurality of images from one or more subjects;
training a model using at least one machine learning technique based on the plurality of historical masks;
generating, using the trained model, the mask for the location according to the plurality of historical masks; and
refining the mask based on a comparison between the generated mask and the plurality of historical masks associated with the location for simulating the structure.

10. The method of claim 1, wherein generating the mask comprises determining an appearance of the mask associated with at least an intensity of one or more voxels of the first image.

11. The method of claim 10, wherein determining the appearance comprises:
selecting an aggregated pixel intensity of the mask;
applying at least one pattern for the mask; and
simulating at least one noise for the mask.

12. The method of claim 11, wherein the at least one pattern comprises at least one of: edema pattern, hemorrhagic pattern, necrotic pattern, cystic pattern, inflammatory pattern, tumoral pattern, or ischemic pattern.

13. The method of claim 10, wherein determining the appearance and applying the mask comprises:
providing the generated mask and at least a portion of the first image for applying the mask as inputs to a model trained using a machine learning technique;
generating, using the model, a third image comprising at least the portion of the first image and the generated mask; and
updating the third image based on a comparison of the third image to at least one historical image, the at least one historical image having a second mask at the location with a second shape similar to the shape of the mask.

14. A system for simulating structures in images, comprising one or more processors configured to:
obtain a first image of a subject;
determine a location for simulating a structure within the first image;
simulate, according to the location, a shape for the structure;
generate a mask according to the location and the shape for the structure; and
apply the mask to the first image to generate a second image simulating the structure.

15. A non-transitory computer-readable storage medium storing processor-executable instructions that, when executed by at least one processor, cause the at least one processor to:
obtain a first image of a subject;
determine a location for simulating a structure within the first image;
simulate, according to the location, a shape for the structure;
generate a mask according to the location and the shape for the structure; and
apply the mask to the first image to generate a second image simulating the structure.
